(19) United States
(12) Patent Application Publication
(10) Pub. No.: US 2025/0250218 A1
(43) Pub. Date: Aug. 7, 2025

(54) Anti-Amyloidogenic Curcumin Analogues

(71) Applicant: MCPHS University, Boston, MA (US)

(72) Inventors: Ronny PRIEFER, Hopkinton, MA (US); Michelle YOUNG, Belmont, MA (US); Patrick GORDON, Woburn, MA (US); Greg LANDRY, Brookline, MA (US)

(21) Appl. No.: 19/048,532

(22) Filed: Feb. 7, 2025

Related U.S. Application Data
(60) Provisional application No. 63/550,985, filed on Feb. 7, 2024.

(51) Int. Cl. C07C 49/537; C07K 16/18

(52) U.S. Cl. CPC C07C 49/537; C07K 16/18

(57) ABSTRACT

Extended chalcone compounds with anti-amylogenic activity were prepared and found to lack cytotoxicity and to promote neuroprotection. Testing on an animal model for Alzheimer's Disease revealed improvements in brain function using the high affinity compounds. The compounds inhibited the aggregation of Aβ42 but not its synthesis. The compounds can be used in new therapies for the prevention and treatment of Alzheimer's disease, including in conjunction with antibodies directed at removing amyloid plaques.

### CROSS REFERENCE TO RELATED APPLICATIONS

This application claims the priority of U.S. Provisional Application No. 63/550,985 filed on 7 Feb. 2024 and entitled “Anti-Amyloidogenic Curcumin Analogues”, the whole of which is hereby incorporated by reference.

### BACKGROUND

Alzheimer's Disease (AD) is characterized by the progressive and gradual decline in cognitive function of those afflicted [1]. The damage to the brain may precede the apparent symptoms by up to 20 years [2]. As the disease progresses, symptoms such as depression, mood swings, decreased physical function, and impaired cognition appear, ultimately leading to death [3]. The pathophysiological hallmarks of AD include a reduction in levels of acetylcholine, intracellular flame-shaped neurofibrillary tangles (NFTs) of hyperphosphorylated tau proteins, and extracellular deposits of the amyloid-3 peptide (Aβ), which form senile plaques [4, 5].

One of the main hallmarks of AD is the accumulation of neurotoxic aggregates of Aβ plaques. The Aβ plaques initiate mitochondrial oxidative stress, promoting hyperphosphorylation of tau, resulting in the aforementioned neurotoxicity [5]. The Aβ peptide is derived from the catabolism of the amyloid precursor protein (APP), a type 1 transmembrane glycoprotein present mainly in the neuronal and glial cells of the brain [6]. APP can be catabolized via two alternative pathways: the non-amyloidogenic (physiological conditions) or the amyloidogenic pathway (pathological conditions) [7,8]. In the former pathway, APP proteolysis occurs via the membrane-bound enzyme, α-secretase, to release sAPPα, leaving behind an 83 amino acid C-terminal APP fragment (CTFα) [7]. Further cleavage of the CTFα by γ-secretase yields an extracellular non-toxic soluble fragment (P3) and the APP intracellular domain (AICD). However, under pathological conditions, APP undergoes cleavage by 3-secretase (BACE-1) then γ-secretase at the N and C termini, respectively. When (3-secretase cleaves APP, it leads to the release of sAPPβ and a membrane-tethered, 99 amino acid, C-terminal (CTFβ) unit [9]. Subsequently, CTF is cleaved by γ-secretase, liberating AICD and extracellular Aβ peptides [10,11]. The Aβ monomers can aggregate into oligomers to form insoluble fibrils, which further arrange themselves into a β-sheet conformation. The β-sheets cumulatively form ordered fibrils, known as β-plaques [12]. The presence of β-amyloid plaques in the brain marks a characteristic feature of AD. These plaques interfere in neuronal signaling, which disrupts brain cell functions. The result is a loss of neuronal synapses, progressive decline in neurotransmitter activity, inflammation, and neuronal cell death [13]. Additionally, Aβ can affect microtubule stability by increasing Ca²⁺ influx and activating protein kinases, leading to the aforementioned tau hyperphosphorylation [14, 15].

Amyloid inhibitor therapies have been attempted to reduce Aβ peptide production, either via α-secretase stimulation, inhibition of γ-secretase, and/or inhibition of β-secretase [16]. Another approach being taken is to develop amyloid antiaggregant therapies, either to inhibit Aβ fibrillation and/or oligomerization, or to clear out existing plaques. Aducanumab is a human immunoglobulin gamma 1 monoclonal antibody targeting Aβ oligomers [17]. More recently, lecanemab and donanemb have come into use, both of which bind to and remove the aggregated Aβ fibrils [18].

Currently, there is no small molecule treatment for AD, nor is there a way to stop the progressive death of neuronal cells associated with this devastating aliment. The antibodies mentioned above only temporarily slow progression of AD, and do not interfere with its pathogenesis. Previous research suggests that homotaurine and curcumin may have the ability to inhibit Aβ plaque formation. Homotaurine is a naturally occurring small molecule found in some seaweeds, and curcumin is a phenolic antioxidant found in Curcuma longa. It has been suggested that curcumin may act on AD by causing Aβ disaggregation, by an anti-inflammatory effect, and/or by acting as an antioxidatant.¹³ In vitro studies have shown that curcumin inhibits Aβ₄₀ aggregation and prevents Aβ₄₂ oligomer formation at concentrations between 0.1 and 1.0 μM, but appears to have low permeability across the blood brain barrier.¹⁴ Thus, there remains a need for small molecule drugs that inhibit Aβ plaque formation and are able to cross the blood-brain barrier.

### SUMMARY

The present technology provides prevention of the formation of amyloid-β plaques by small molecules. In view of the success of antibodies in clearing amyloid-β (Aβ), small molecules that have such anti-amyloidogenic activity against Aβ plaques may offer a promising and more attractive option. Small molecule drugs can be employed, for example, as a management drug administered after or in conjunction with antibodies used for plaque clearance. Such a treatment regimen may improve on currently available treatments and assist a wide range of AD patients.

The inventors have discovered that certain curcumin analogues display low μM inhibition of plaque formation (FIG. 1A, Compounds 1-3). It was found that having a dialkylamino substituent on Ring B was needed for effectiveness (see FIG. 1B and [5]). Only a small library of compounds with varying substituents on Ring A was assessed in this early study. More recently, extended chalcone analogs were synthesized with modifications on both A and B rings (FIG. 1B). These were tested using the Thioflavin T (TfT) fluorescence assay to measure their ability to inhibit Aβ aggregation. The most active compounds were further evaluated for potential neurotoxicity and neuroprotection using a cell viability assay. These compounds were non-neurotoxic and displayed effectiveness at neuroprotection. Compounds were evaluated in a Drosophila melanogaster animal AD model. The forced climbing assay revealed statistically significant changes in flies' movement by ˜30% when fed the extended chalcone anti-amyloidogenic agents.

One aspect of the present technology is a small molecule treatment for AD that can be employed either before of after Aβ clearance from the brain using antibodies against Aβ; with the latter targeted at the prevention of the re-emergence of the disease.

The technology can be further summarized in the following listing of features.

1. A compound of any of Formulas I-V below:

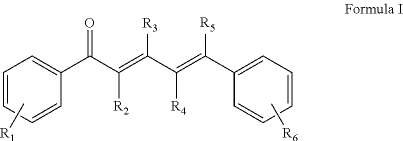

wherein
R1 is one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof;
R2, R3, R4, and R5 are independently hydrogen or a substituent; and
R6 is dialkylamino with each alkyl portion having C1-C5, or the alkyl portions fused to form a 5- or 6-membered saturated ring, or hydrogen;

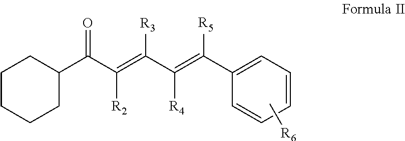

wherein
R2, R3, R4, and R5 are independently hydrogen or a substituent; and
R6 is dialkylamino with each alkyl portion having C1-C5, or the alkyl portions fused to form a 5- or 6-membered saturated ring, or hydrogen;

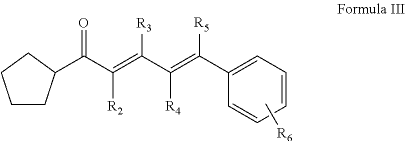

wherein
R2, R3, R4, and R5 are independently hydrogen or a substituent; and
R6 is dialkylamino with each alkyl portion having C1-C5, or the alkyl portions fused to form a 5- or 6-membered saturated ring, or hydrogen;

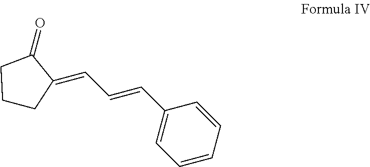 [SMILES: O=C1CCC/C1=C\C=C\c1ccccc1]

wherein any position not indicated with H can be independently any substituent;

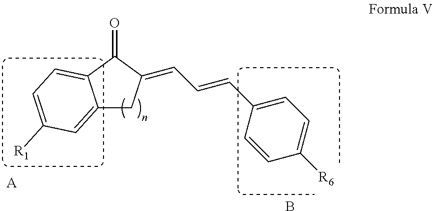

wherein
R1 is one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, 01-05 keto, 01-05 thioether, C1-C6 alkyl or cycloalkyl, hydrogen, another substituent, or any combination thereof;
R6 is dialkylamino with each alkyl portion having 01-05, or the alkyl portions fused to form a 5- or 6-membered saturated ring, or hydrogen; and
n=0, 1, 2, or 3.
   2. The compound of feature 1, wherein the compound is a compound of Formula VIII below:

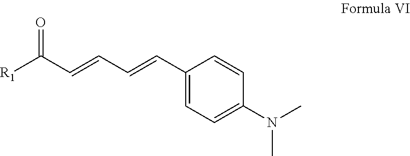

wherein R1 is an aromatic ring with one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof.
   3. The compound of feature 2, wherein the compound is selected from the group consisting of Compounds 4-7, 9-10, and 13-28.
   4. The compound of feature 3, wherein the compound is selected from the group consisting of Compounds 4-5, 7, 13-14, 18-22, and 25-27.
   5. The compound of feature 1 or feature 2, wherein the compound is not any of Compounds 6, 9, 10, 15, 16, 17, 23, 24, or 28.
   6. The compound of feature 1, wherein the compound is selected from the group consisting of Compounds 32-34.
   7. The compound of any of the preceding features, wherein the compound inhibits aggregation of an amyloid precursor, such as Aβ 1-42.
   8. The compound of any of the preceding features, wherein the compound inhibits amyloid plaque formation in a mammalian brain, such as in a human brain.
   9. The compound of any of the preceding features which can cross a blood-brain barrier of a living mammal.
   10. A pharmaceutical composition comprising a compound of any of the preceding features and one or more excipients.
   11. A method to aid in preventing formation or re-formation of amyloid plaque in a mammalian subject's brain, the method comprising administering an effective amount of a compound of any of features 1-9 or the pharmaceutical composition of feature 10 to the mammalian subject.
   12. The method of feature 11, wherein the subject was previously treated to remove amyloid plaques from its brain.
   13. The method of feature 12, wherein the previous treatment comprises administration of an antibody having a binding specificity for an epitope found in amyloid plaque.
   14. The method of feature 13, wherein the antibody is selected from the group consisting of aducanumab, lecanemab, and donancinab.
   15. The method of any of features 11-14, wherein amyloid plaque formation in the subject's brain is inhibited to any extent.
   16. The method of any of features 11-15, wherein the subject is a human patient who has Alzheimer's Disease.
   17. A method to aid in treating Alzheimer's Disease, the method comprising:
(a) administering to a human patient who has Alzheimer's Disease an antibody having a binding specificity for an epitope found in amyloid plaque; and
(b) administering an effective amount of a compound of any of features 1-9 or the pharmaceutical composition of feature 10 to said patient.
   18. The method of feature 17, wherein steps (a) and (b) are performed sequentially or concurrently.
   19. The method of feature 17 or feature 18, whereby reoccurrence of amyloid plaque formation in the patient is inhibited compared to a method comprising only step (a) of feature 17.
   20. The method of any of features 17-19, wherein the antibody administered in step (a) is selected from the group consisting of aducanumab, lecanemab, and donancinab.
   21. A kit comprising:
(i) a compound of any of features 1-9 or the pharmaceutical composition of feature 10; and
(ii) an antibody having a binding specificity for an epitope found in amyloid plaque.
   22. The kit of feature 21, where the antibody is selected from the group consisting of aducanumab, lecanemab, and donancinab.

As used herein, a “substituent”, unless otherwise specified, can be any of the following: hydrogen, hydroxy, sulfoxy, halo, acyl, acyloxy, alkyl, heteroalkyl, alkenyl, heteroalkenyl, alkynyl, heteroalkynyl, alkoxy, cycloalkyl, heterocycloalkyl, aryl, arylalkyl, arylhalo, arylhydroxy, arylcyano, aryltrifluoromethyl, aryltrifluoromethoxy, aryinitro, aryltrifluoromethoxy, arylnitro, and arylether, arylester, arylsulfonyl, arylsulfinyl, arylsulfonamidyl, arylsulfonate, arylsulfoxyl, arylphosphate ester, arylcarbonyl, arylcarboxylate, arylcarbamate, arylamine, arylimide, heteroaryl, heteroarylalkyl, heteroarylhalo, heteroarylhydroxy, heteroarylcyano, heteroaryltrifluoromethyl, aryltrifluoromethoxy, aryinitro, heteroaryltrifluoromethoxy, heteroaryinitro, and heteroarylether, heteroarylester, heteroarylsulfonyl, heteroarylsulfinyl, heteroarylsulfonamidyl, heteroarylsulfonate, heteroarylsulfoxyl, heteroarylphosphate ester, heteroarylcarbonyl, heteroarylcarboxylate, heteroarylcarbamate, heteroarylamine, heteroarylimide, quinidine, morpholine, and any ring structure is optionally substituted with any of the substituents described herein, with the proviso that any two adjacent substituents can come together to form a carbocyclic or heterocyclic ring system. A hydrocarbon or heterocyclic ring system can be phenyl, thienyl, furanyl, pyrimidinyl, oxazoyl, thiazolyl, pyridyl, naphthyl, quinolinyl, indolyl, benzothiophenyl, benzofuranyl, pyrrolyl, imidazolyl, pyrazole, triazolyl, isoxazolyl, pyridazinyl, pyrazinyl, pyrimidinyl, oxadiazolyl, benzimidazolyl, or triazinyl. A heterocyclic ring system may contain one or more heteroatoms selected from the group consisting of oxygen, sulfur, nitrogen, and combinations thereof.

### BRIEF DESCRIPTION OF DRAWINGS

FIG. 1A shows IC₅₀ values of some extended chalcone derivatives. FIG. 1B shows areas of an extended chalcone scaffold where modifications were made.

FIG. 2 shows a synthetic scheme (Scheme 1) for extended chalcone analogs Compounds 4-28 and 31-34).

FIG. 3 shows results of a Thioflavin T fluorescence assay screening for Compounds 11, 20, 22, 23, and 24 at 10 μM and Phenol Red (100 μM) as positive control.

FIGS. 4A-4D show structures and IC₅₀ values of an extended chalcone library with Ring A modifications.

FIG. 5A shows Scheme 2 for synthesis of cinnamaldehyde 29 and 30 by acid catalyzed condensation. FIG. 5B shows structures and IC₅₀ values of an extended chalcone library with Ring B modifications.

FIG. 6 shows results of a cell viability assay measured as an MTT reduction. Data are shown as +/−SEM (n=3), normalized as % vehicle control. Asterisks indicate significant difference from vehicle controls determined by a one-way ANOVA followed by Tukey's post hoc test (****p<0.0001, **p<0.001, *p<0.05).

FIG. 7 shows a representative Punnett square of the cross between UAS-Aβ 1-42 and nSyb-Ga/4 with the TM3, Sb balancer on the 3^{rd} chromosome.

FIG. 8 shows the percentage climbing for compounds 25, 33, and 34, compared to control, representing the percentage of flies in each section of the vial after 10 s, with standard deviation. Statistical significance is indicated with $ for the top, # for the middle, and * for the bottom. Single symbol indicates a p<0.05, double indicates a p<0.001, and triple indicates a p<0.0001. Compound 25: 1 mcg/mL bottom 95% CI [1.543-24.82]p=0.0223, middle 95% CI [−18.97-−3.487]p=0.0024. Compound 33: 1 mcg/mL bottom 95% CI [18.63-40.04]p=<0.0001, middle 95% CI [−29.70-−13.63]p=<0.0001, and top 95% CI [−12.8-−2.516]p=0.0018, 10 mcg/mL bottom 95% CI [10.63-32.04]p=<0.0001, middle 95% CI [−26.04-−9.964]p=<0.0001. Compound 34: 10 mcg/mL bottom 95% CI [20157-23.51]p+0.0142, middle CI [−18.78-−2.558]p=0.0065, 100 mcg/mL bottom 95% CI [3.395-24.75]p=0.0064, middle 95% CI [−18.67-−2.463]p=0.0071.

### DETAILED DESCRIPTION

The present technology provides libraries of chalcones and extended chalcone analogs. The library of extended chalcones has some structural similarities to curcumin, but lacks the β-dicarbonyl moiety which hinders the bioavailability of curcumin. Using fluorescence spectroscopic analysis with Thioflavin T (TfT), the anti-aggregation effect of these compounds on Aβ₄₂ was determined. A number of the compounds were identified as potential anti-amyloidogenic agents, and those were further evaluated with regard to their neurotoxicity and neuroprotection. The extended chalcone analogs that displayed the most anti-aggregation effect on Aβ₄₂ were further analyzed using a cell viability assay, and none of the compounds tested displayed any neurotoxicity. Some of the compounds were tested using a transgenic Drosophila melanogaster model of AD (nSyb-Gal4 crossed with UAS-Af342), and no adverse effects on life span were found. Treatment groups performed better on a forced climbing assay by up to 68% (a statistically significant improvement) when fed an extended chalcone, indicating improved locomotor activity, and thus a potential anti-amyloidogenic agent for AD.

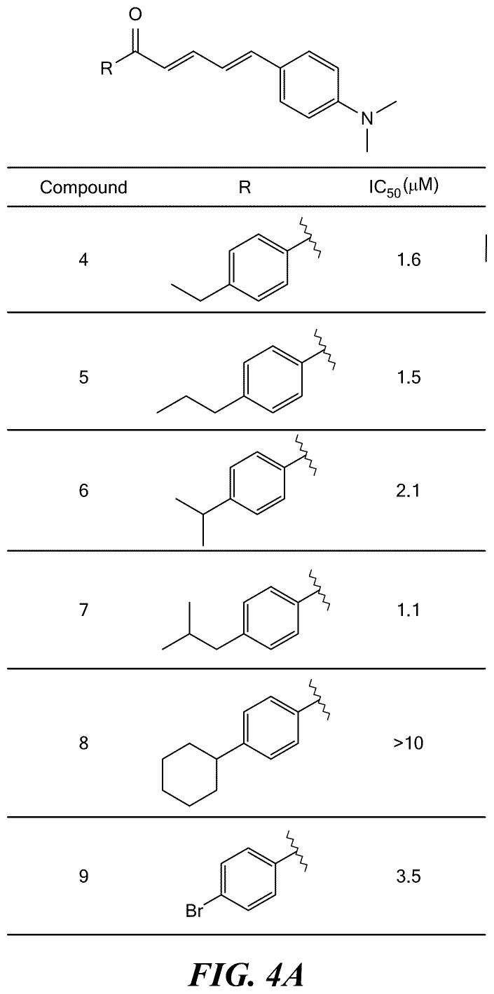

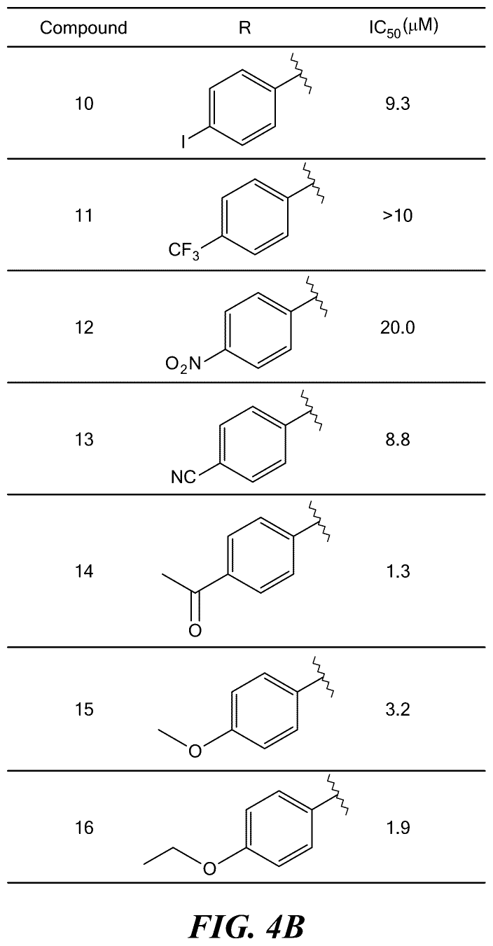

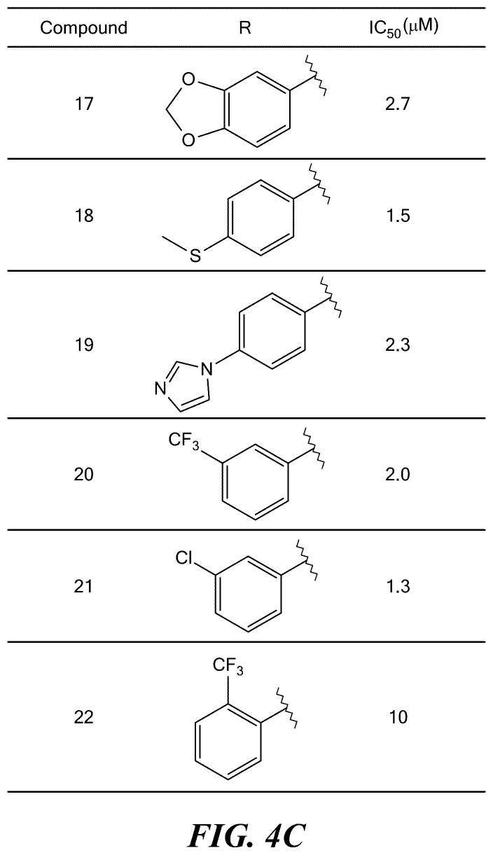

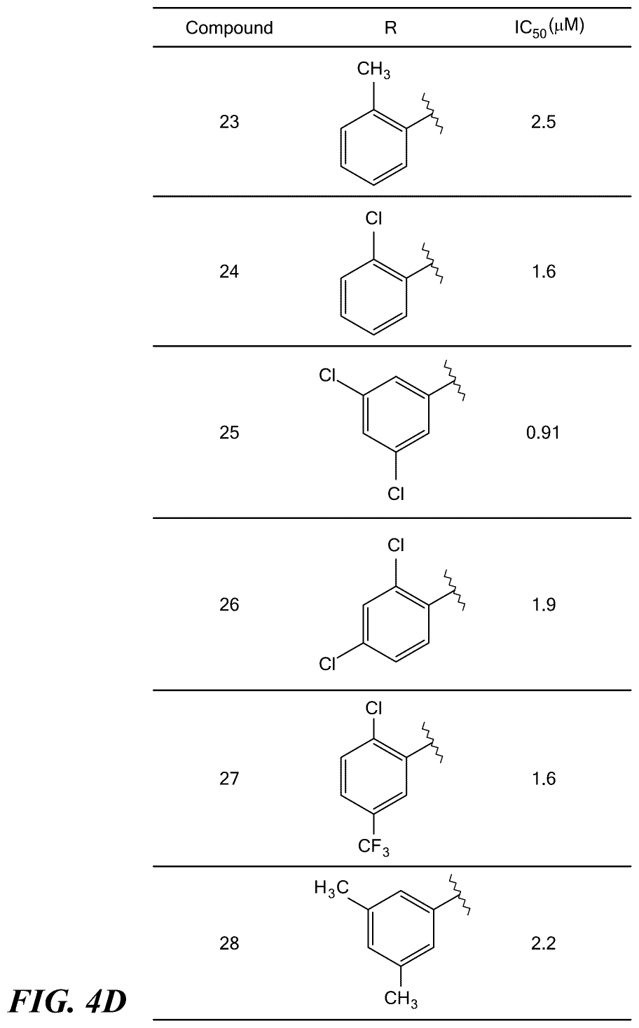

An embodiment of the present technology is an extended chalcone compound of Formula I as shown below. A preferred substituent for R6 is a para-dimethylamino group. Preferred embodiments of phenyl-R1 are shown in FIGS. 4A-4D. R2, R3, R4, and R5 are preferably hydrogen.

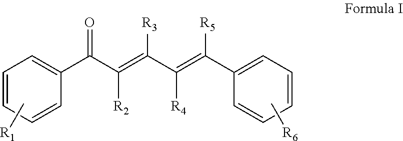

For Formula I, R1 is one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof; R2, R3, R4, and R5 are independently hydrogen or a substituent; and R6 is dialkylamino with each alkyl portion having C1-C5, or the alkyl portions fused to form a 5- or 6-membered saturated ring, or hydrogen.

Another embodiment is a compound according to Formula II below. A preferred substituent for R6 is a para-dimethylamino group, and R2, R3, R4, and R5 are preferably hydrogen.

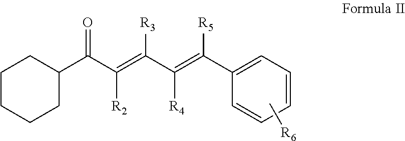

For Formula II, R2, R3, R4, and R5 are independently hydrogen or a substituent; and R6 is dialkylamino with each alkyl portion having C1-C5, or the alkyl portions fused to form a 5- or 6-membered saturated ring, or hydrogen.

Yet another embodiment is a compound according to Formula III below. A preferred substituent for R6 is a para-dimethylamino group, and R2, R3, R4, and R5 are preferably hydrogen.

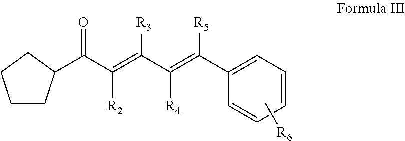

For Formula III, R2, R3, R4, and R5 are independently hydrogen or a substituent; and R6 is dialkylamino with each alkyl portion having C1-C5, or the alkyl portions fused to form a 5- or 6-membered saturated ring, or hydrogen.

Still another embodiment is a compound according to Formula IV below.

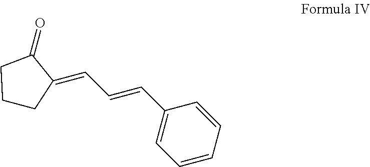 [SMILES: O=C1CCC/C1=C\C=C\c1ccccc1]

For Formula IV, any position not indicated with H can be independently any substituent.

Another embodiment is a compound according to Formula V below. A preferred substituent for R6 is a para-dimethylamino group.

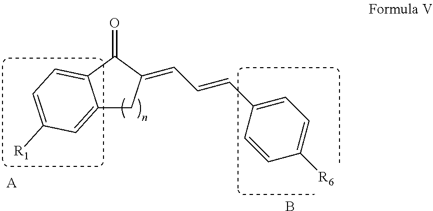

For Formula V, R1 is one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof; R6 is dialkylamino with each alkyl portion having C1-C5, or the alkyl portions fused to form a 5- or 6-membered saturated ring, or hydrogen; and n=0, 1, 2, or 3.

Yet another embodiment is a compound according to Formula VI below.

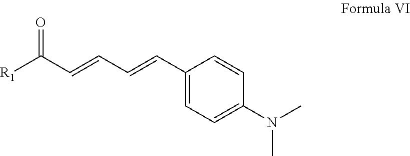

wherein R1 is an aromatic ring with one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof.

The compounds disclosed herein have advantages over previous treatments of AD in that they can prevent plaque buildup. Antibody treatments are directed to removing already formed plaque. The compounds described herein do not remove plaques, but they can prevent their buildup. The compounds could be used, for example, to prevent re-occurrence of plaques in patients who suffer from AD and are treated with, or have been treated with, have shown improvement with aducanumab, lecanemab, or donanemab, or other antibodies or antibody-like biotherapeutics that may be developed to cause the immune system to remove already formed plaque.

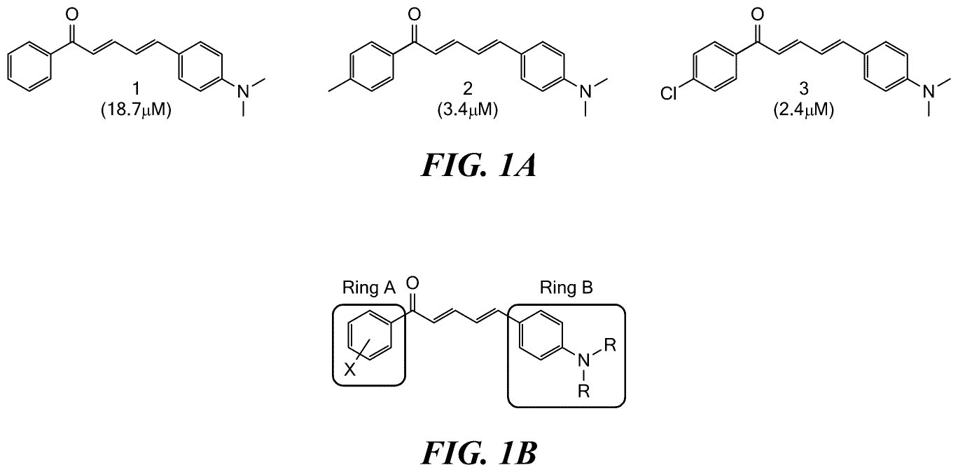

Among the initial set of synthesized extended chalcones, Compounds 1, 2, and 3 were found to have the lowest IC₅₀ values as anti-amyloidogenic compounds (FIG. 1A). In order to expand upon the library of compounds, analysis of the substitution and pattern on Ring A was carried out, with some subsequent analysis of the amino group on Ring B (FIG. 1B). Analogs were designed to maintain an electron donating group on Ring B, while with Ring A different substituents at varying positions were synthesized to assess the electronics and steric constraints.

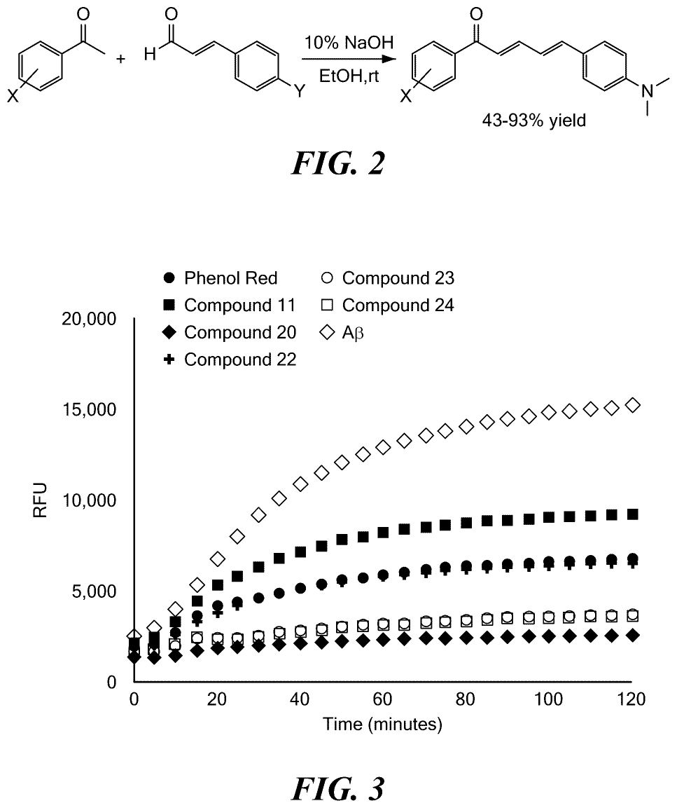

The library of compounds with different substituents on Ring A were synthesized using an aldol condensation reaction carried out between acetophenone derivatives and 4-(dimethylamino)cinnamaldehyde dissolved in ethanol, using 10% NaOH as the base (Scheme 1, FIG. 2). The product precipitated out of the solution as the reaction progressed. After 24 h, ice chips were added to the reaction mixture to increase precipitation and improve the yield. The solid was collected by vacuum filtration and washed with a mixture of cold water and ethanol to produce the desired extended chalcone in good to excellent yields, without any need of further purification. Some reactions produced oils rather than solids. In those cases, the oil was concentrated and purified using flash column chromatography. ¹H-NMR was utilized to confirm the success of reactions, as well as their purity.

The acetophenone derivatives used had varying electronic and steric effects to probe the structure activity relationship. To study the effect of lipophilicity, alkyl analogs were synthesized ranging from methyl to isopropyl, isobutyl, and cyclohexyl groups at the para position (see FIGS. 4A-4D). Additionally, to investigate the electronic effects of substituent nature on Ring A, both electron withdrawing groups and electron donating groups were incorporated at the para position. The electron withdrawing groups included nitro, cyano, and acetyl substituents, while the electron donating groups included methoxy, ethoxy, and methylthio groups. Halogens, as well as trifluoromethyl analogs, which impacted both solubility and electronics, were synthesized. To explore the effects of position of the substituents, chloro and trifluoromethyl group analogs were additionally synthesized at the ortho and meta position of Ring A. To further explore the electronic and steric effects, a small number od di-substituted extended chalcones were also synthesized.

All newly synthesized compounds were screened for their anti-amyloidogenic activity on Aβ1-42 peptides, using Phenol Red as the internal standard—all at 100 μM. The compounds were significantly superior to control, necessitating the need to lower their concentration to 10 μM and compare to Phenol Red at 100 μM (FIG. 3). To obtain the IC₅₀ values (shown in FIGS. 4A-4D), all compounds were tested at a minimum of five different concentrations.

Comparing Compounds 1, 2, 4, and 5, upon increasing the length of the alkyl substituent (—H, —CH₃, ethyl, n-propyl) and subsequent c log P values [19], a decrease in the IC₅₀ value was observed, suggesting that as lipophilicity increases, anti-aggregation activity of the compound improves. Comparing Compound 5 to Compound 6, going from n-propyl to i-propyl, a slight decrease in activity was observed. However, Compound 8 with the cyclohexyl substituent and a c log P value of 5.59 [19], displayed a reduction in anti-aggregation activity. Thus, a lipophilic, unbranched substituent was favorable.

Halogen substituents also improved aggregation inhibition in comparison to the unsubstituted extended chalcone (Compound 1). The p-chloro analog (Compound 3) had the highest activity compared to the other halogen derivatives, followed by p-bromo (Compound 9), and lastly p-iodo (Compound 10). Since Compounds 3, 9, and 10 have similar lipophilicities, the decrease in activity may be a result of the decrease in electronegativity. Other highly electron-withdrawing groups such as trifluoromethyl (Compound 11) and cyano (Compound 13) at the para position, although better than the unsubstituted chalcone (Compound 1), displayed higher IC₅₀ values than many others.

The evaluation of electron donating groups was possible when comparing Compound 15 to Compound 16. The latter, with an ethoxy group, displayed better anti-amyloidogenic activity than the former, containing a methoxy substituent. As indicated above, increasing lipophilicity increased activity, up to a certain point. Switching from an oxygen atom (Compound 15) to sulfur (Compound 18), led to a drop in the IC₅₀ value, from 3.2 μM to 1.5 μM. This could be owed to the progressive increase in lipophilicity, suggesting a p-ethylthio-containing analog would be potent.

To test the effect of the substituent position, m-trifluoromethyl (Compound 20), o-trifluoromethyl (Compound 22), m-chloro (Compound 21), and o-chloro (Compound 24) containing analogs were synthesized. For both substituents, the meta substituted analog had a noticeable decrease in its IC₅₀ value. Comparing para vs. ortho substituted compounds, Compound 2 (p-methyl) versus Compound 20 (o-methyl), the ortho substitutions displayed better activity. From these data, it appears that the ideal mono substituted position is meta>ortho>para.

The effect of di-substituted derivatives on Ring A was studied. Compounds 25 and 26, both di-substituted chloro analogs, showed comparable or better activity than their mono-substituted counterparts (i.e., Compounds 3, 21, and 24). The 3,5-dichloro analog (Compound 25) had the lowest IC₅₀ value (0.91 μM) of all the Ring A modified analogs. This suggests that di-substitution at the meta-position lead to significant improvement in anti-aggregation activity. Collectively, these data suggest that increasing lipophilicity leads to an improvement in anti-aggregation activity of extended chalcones, up to a certain point. Furthermore, di-substituted extended chalcones could lead to further enhancement of activity.

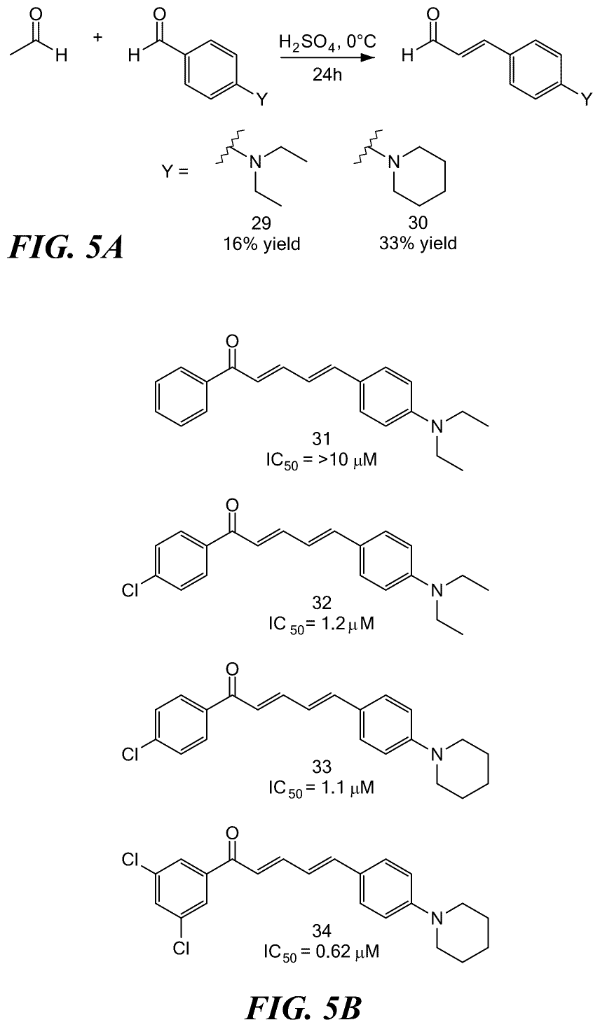

Next, modifications on Ring B were made to add more lipophilic groups while maintaining the electron donating nature of the amino substituent. It was thus necessary to first synthesize para-amino substituted cinnamaldehydes. Initially, a Wittig reaction was attempted; however, a lower than ideal yield was produced. Ultimately, an acid catalyzed aldol condensation reaction was used for the synthesis of cinnamaldehyde derivatives (Scheme 2, FIG. 5A). This acid catalyzed condensation reaction involved initially dissolving a p-aminobenzaldehyde in concentrated H₂SO₄ under vigorous stirring and cooling to 0° C. The acetaldehyde was then added dropwise over 4 h. The reaction mixture was poured over ice and brought to an alkaline pH (20% NaOH). The product was extracted with dichloromethane and purified by flash chromatography to obtain the pure product in moderate yields. ¹H-NMR was utilized to confirm the success of the reactions as well as their purity. To explore the effects of increasing the alkyl chain length, 4-(diethylamino)cinnamaldehyde (Compound 29) and 4-(piperidin-1-yl)cinnamaldehyde (Compound 30) were synthesized and further coupled, using the aforementioned aldol condensation (Scheme 1, FIG. 2) with acetophenone, 4′-chloroacetophenone and 3′,5′-dichloroacetophenone to yield the extended chalcones (Compounds 31-34, FIG. 5B).

All four compounds were evaluated for their IC₅₀ values, which are shown in FIG. 5B. Similar to the Ring A modification library, upon increasing the c log P value from 4.45 for Compound 3, to 5.07 and 5.13 for Compounds 33 and 32, respectively, a decrease in IC₅₀ values was observed from, 2.4 to 1.1 to 1.0 μM. This followed the observed trend that suggested a correlation between lipophilicity and anti-amyloidogenic activity. The 3,5-dichloro analog (Compound 25), which showed the lowest IC₅₀ value (0.91 μM) of all Ring A modifications, ultimately produced the most effective anti-amyloidogenic compound when coupled with 4-(piperidin-1-yl)cinnamaldehyde, specifically Compound 34, with an IC₅₀ value of 0.62 μM.

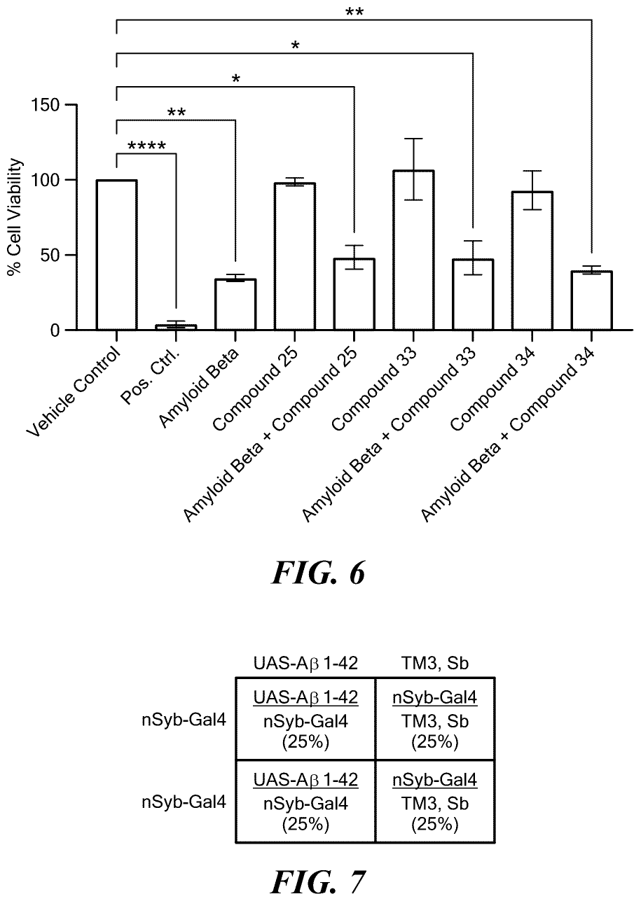

The cell viability and cytotoxicity was examined for the compounds that displayed the greatest anti-amyloidogenic activity. The 3-(4,5-dimethylthioxzol-2-yl)-2,5-diphenyltretazolium bromide (MTT) assay was used to assess the viability of SH-SY5Y cells when exposed to Compounds 25, 33, and 34 at their IC₅₀ values (FIG. 6). Aβ42 alone led to an approximately 65% decrease in cell viability, corresponding with its well-known neuro-cytotoxicity. All three compounds alone showed no cytotoxicity when administered at their corresponding IC₅₀ value concentrations. When the extended chalcones (again at their IC₅₀ value concentrations) were administered concurrently with Aβ42, a trend toward improvements in cell viability was observed. Notably, the Aβ42-induced cytotoxicity in SH-SY5Y cells after 48 h went from a 65% decrease in cell viability to only a 52, 52, and 60% decrease, when co-administered with Compounds 25, 33, and 34, respectively.

As the compounds tested displayed notable anti-amyloidogenic activity and no cell cytotoxicity, but no statistically significant neuroprotection when co-administered with Aβ42, their in vivo activity was next tested. These compounds were initially tested on an AD-modeled, genetically modified Caenorhabditis elegans (C. elegans, CL2006) [20]. These C. elegans model organisms are predisposed to developing amyloid plaques. Compounds were tested at multiple concentrations and the C. elegans mobility was observed after different time points when exposed to extended chalcones. The C. elegans displayed notable, partial, or complete, reversal of the physiological AD marker, specifically, a lack of mobility. Due to these encouraging results, further in vivo work was focused on a Drosophila melanogaster model that overexpressed Aβ.

To produce the transgenic Drosophila line of interest, UAS-Aβ42 1-42 female virgins were crossed with nSyb-Gal4 males. UAS-Aβ42 1-42 has a TM3, Sb balancer on the third chromosome, resulting in the mutant Sb gene, indicative of stubble bristles as a dominant marker [21]. When crossed with the driver line, such as nSyb-Gal4, the transgenic line will either be UAS-Aβ42 1-42/nSyb-Gal4 or nSyb-Gal4/TM3, Sb. The transgenic line of interest is the F1 progeny that expressed Aβ42 1-42 peptide (UAS-Aβ42 1-42/nSyb-Gal4), which have the normal bristle phenotype. All offspring with the inherited balanced nSyb-Gal4TM3, Sb were negatively selected. Once enough F1 progeny of the transgenic line of interest were available, testing of the compounds was carried out.

The extended chalcones of interest were dissolved in DMSO to a concentration of 5 mg/mL. This solution was then further diluted into flyfood with final concentrations of 1, 10, and 100 mcg/mL of fly food, corresponding to 0.0001%, 0.001%, and 0.01% (w/v), respectively. These three concentrations were then introduced to each respective group. The F1 progeny that express Aβ42 peptide (UAS-Aβ 1-42/nSyb-Ga/4), were placed onto the food fortified with the different concentrations of Compound 25, 33, or 34 for 15 days.

Initially, a lifespan assay was performed. Wild Type¹¹¹⁸ Bonini flies were placed into corresponding vials of food containing the compounds at the various concentrations. Over the course of the 15 days, both wild type and the transgenic line flies were counted in each vial to determine how many living flies remained. The flies were tipped onto new food with the same compound and concentration three times during the course of the study to avoid potential deaths due to the food becoming too moist from larvae being laid. After the 15 days, the total number of living flies were counted, and a Kaplan-Meier survival was generated. As anticipated, no statistically significant changes in survival between the WT and transgenic line were observed, due to the lack of neurotoxicity previously observed for these compounds.

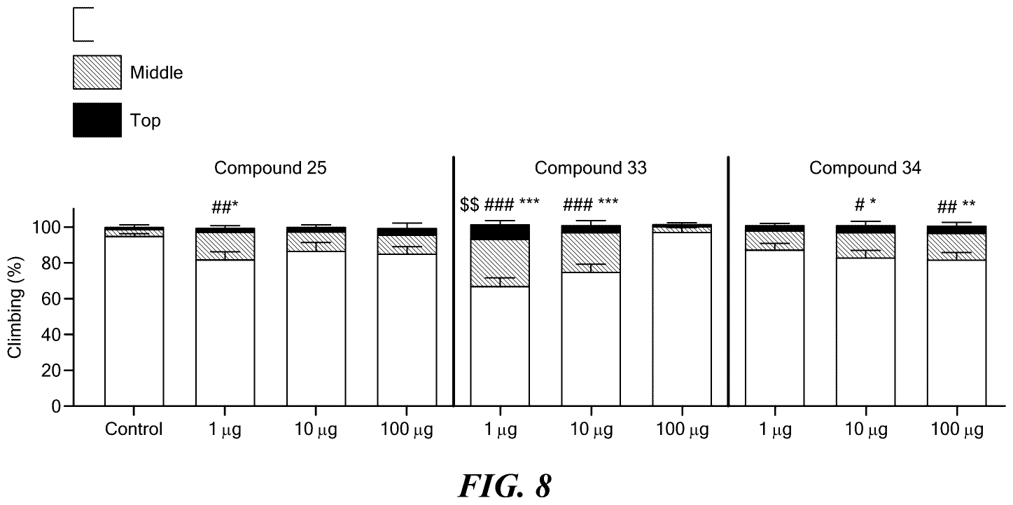

A forced climbing assay was conducted on the transgenic flies exposed to the extended chalcone compounds. Flies that overexpressed Aβ should exhibit age dependent defects in climbing [22]. Conversely, flies that did not overexpress Aβ should exhibit negative geotaxis, allowing them to climb to the top of the vial in 10 s or less. The climbing assay was conducted five times a day, beginning on the seventh day after the flies were exposed to the compounds. Vials were marked with top, middle, and bottom sections, separated by one inch. Flies had 10 s to climb after being tapped to the bottom. The percent total in each section, for each trial, was calculated and a one-way ANOVA was preformed to compare the percentage of flies in the top, middle, and bottom of the control vs. the transgenic flies when exposed to Compounds 25, 33, and 34 at various concentrations (FIG. 8).

Compound 25 at 1 mcg/mL exhibited a statistically significant change in the percentage of flies in the bottom and middle when compared to the control. For example, in the control group, the bottom section contained approximately 95% of the flies, while flies fed Compound 25 saw a drop to 82.15%. This change corresponded to an increase in flies in the middle section, from 4.33% to 15.56%, for the control and Compound 25, respectively. Transgenic flies fed Compound 33 at 1 mcg/mL also showed a statistically significant change in the percentage in each section of the vial, with the bottom containing 66.00%, middle 26.00%, and the top an impressive 8.00% (compared to the control with 0.33%). Compound 33 also showed statistical significance improvements at 10 mcg/mL (bottom: 74.00% and middle: 22.33%). Finally, Compound 34 showed statistically significant improvements at both 10 and 100 mcg/mL, with 82.50% and 81.26% of the flies in the bottom section and 15.00% and 14.90% in the middle, respectively.

The forced climbing assay results indicate that several of the extended chalcones exerted a physiological effect on the flies, with Compound 33 at 1 mcg/mL displaying the greatest effect. Although a larger portion of the flies, for all compounds and concentrations, still remained in the bottom section of the vials, this climbing improvement showed a clear physiological benefit on the transgenic flies that overexpress Aβ.

To ensure that the compounds only inhibited the formation of the plaques and not the production of Aβ, an enzyme-linked immunosorbent assay (ELISA), using the Human A(342 ELISA Kit (Invitrogen: #KHB3441) was performed to quantify the levels of Aβ. The treated and control transgenic flies previously used for the climbing assay were decapitated, and the heads were split among four tubes, to which 100 mcL of lysis buffer was added. Heads were then homogenized using sonication for 30 min, and frozen (−18° C.) until the ELISA was performed.

To perform the ELISA, the samples were first thawed on ice. A protease inhibitor cocktail with AEBSF (a serine protease inhibitor) was added to each sample, with a final concentration of 1 mM, and were then centrifuged to remove the large particulate matter. After centrifugation, samples were diluted with Standard Diluent Buffer at a 1:50 ratio to ensure sample concentrations would be within the range of the standard curve. A serial dilution with native human Aβ42 (Hu A(342) was performed in the Standard Diluent Buffer to generate a standard curve. To bind the antigen, 50 mcL of the standard, sample, and control was added to the appropriate wells, with duplicates of each, plus 50 mcL of the detector (Hu Aβ42 Detection Antibody solution). The plate was covered and incubated at room temperature with shaking. After 3 h, the solution was thoroughly aspirated, and the wells were washed four times with 1× Wash Buffer. Anti-Rabbit IgG HRP (100 mcL) was added to every well, and the plate was covered and incubated at room temperature. After 30 min, the solution was thoroughly aspirated, and the wells were washed four times with Ix Wash Buffer. Stabilizing Chromogen (100 mcL) was added to each well and the plate was incubated in the dark. After 30 min, 100 mcL of the Stop Solution was added to every well and the plate was read immediately at an absorbance of 450 nm.

Absorbance measurements for each sample and control were averaged, and from the calibration curve the corresponding level of AB was determined. Data were analyzed using a one-way ANOVA. Regardless of what compound was fed to the flies, and at what concentration, no statistically significant change in the levels of AB was observed. This result supports the conclusion that the tested compounds worked by inhibiting plaque formation, as opposed to inhibiting Aβ42 generation. Thus, the physiological benefit observed when the flies were fed either Compound 25, 33, or 34 at varying concentrations, was most likely a result of a decrease in brain plaque burden.

In summary, twenty-two modifications were made to Ring A of the molecular scaffold shown in FIG. 1B, and four to Ring B. Generally, electron donating groups were found to be superior to electron withdrawing groups on Ring A. Upon increasing the lipophilicity, the anti-aggregation activity also increased, up to a certain point. The meta-position was found to be ideal for mono-substitution on Ring A. Compounds that were di-substituted on Ring A at the meta-positions displayed superior activity. Increasing the lipophilicity of the electron donating group on the para position on Ring B led to an improvement in the anti-aggregation activity. Compounds 25, 33, and 34, which displayed the greatest anti-amylogenic activity, were tested for their toxicity and neuroprotection. None were deemed toxic, and all trended toward displaying neuroprotection. Subsequent testing on an animal model revealed that there were statistical improvements in the forced climbing assay with these compounds. The compounds inhibited the aggregation of Aβ42 but not its synthesis.

### EXAMPLES

### Example 1. Materials and Methods

All reagents were purchased from Millipore Sigma (Burlington, VT, USA) and used without further purification. All synthesized compounds were purified using flash column chromatography. ¹H-NMR and ¹³C-NMR were recorded at 400 MHz on a Bruker Topspin 4.2.0 instrument (Bruker Corp., Billerica, MA, USA). EA was performed on a Carlo Erba 1108 elemental analyzer (Thermo Fisher Scientific, Waltham, MA, USA), performed by Atlantic Microlab, Inc. (Norcross, GA, USA). Compounds 6 [23], 9 [24], 10 [24], 15 [25], 16 [26], 17 [27], 23 [28], 24 [23], and 28 [28] were previously reported and compared for authenticity. For in vitro studies, a fluorometric assay was performed in 96 non-binding microplates from Greiner Bio-One with a clear bottom on a Synergy Bio-tek HTS plate reader (Agilent Technologies, Lexington, KY, USA).

### In Vitro Assays

To test the anti-aggregation effects of the compounds, a SensoLyte, Thioflavin T β-Amyloid (1-42) aggregation kit was used and performed as described [8]. To test neurotoxicity and neuroprotective effects of the compounds, a 3-(4,5-dimethylthiazol-2-yl)-2,5-diphenyltetrazolium bromide (MTT) assay was performed using compounds 25, 33, and 34 as described [8].

### Thioflavin T (ThT) Fluorescence Assay

Samples of ThT (final concentration of 200 μM) and Aβ₁-42 peptide (final concentration of 35 μM) were incubated at 37° C. in a black clear bottom 96-well plate. The ThT fluorescence intensity of each sample was immediately measured every 5 min for 120 min, with 440/485 nm excitation/emission filters and with 15 sec shaking between reads to facilitate aggregation. An inhibitor control containing Aβ1-42 and a supplied aggregation inhibitor (Phenol Red), at a final concentration of 100 μM. The positive control contained Aβ1-42 without an inhibitor. The vehicle control contained the assay buffer and DMSO of concentrations that did not exceed 1%. The tested compound wells contained Aβ42 peptide and the extended chalcone derivatives at varying concentrations. All the wells were brought to 100 μL as a final volume.

### Cell Viability

### Cell Culture and Exposure

SH-SY5Y cells (CRL-2266, ATCC, Manassas, VA) were maintained in SH-SY5Y cells (CRL-2266, ATCC, Manassas, VA, USA) were maintained in Dulbecco's Modified Eagle's Medium and Ham's F-12 Medium (DMEM: F12) supplemented with 50 mL fetal bovine serum (10%) and incubated at 37° C., 5% CO₂, and 90% humidity. For the MTT assay, cells were sub-cultured using trypsin-ethylenediaminetetraacetic acid (EDTA) (0.25%) solution into clear 96-well plates and allowed to adhere for 24 h. Following the removal of growth media, compounds (IC₅₀), Aβ₄₂, at a final concentration of 20 μM, compound+Aβ₄₂, along with positive (500 μM menadione) and vehicle controls were added to designated wells in triplicate. All groups were incubated for 48 h.

### MTT Assay

The MTT assay (Ab211091, Abcam, Waltham, MA, USA) was used to measure cell viability with metabolically active cells reducing the MTT reagent into insoluble formazan. Following the removal of the treatment media, 50 μL of serum-free media and 50 riL of MTT Reagent was added to each well. The plate was then incubated at 37° C. for 3 h. After incubation, 150 μL of MTT Solvent was added into each well. The plate was then wrapped in foil and placed on an orbital plate shaker for 15 min. The absorbance was read at 590 nm.

### Example 2. Aldol Condensation Reactions

### (2E,4E)-5-(4-(dimethylamino)phenyl)-1-(4-ethylphenyl)penta-2,4-dien-1-one (Compound 4)

To a solution of 4′-ethylacetophenone (0.2719 g, 1.83 mmol) in absolute ethanol (10 mL) was added to an aqueous solution of 10% NaOH (5 mL) at 0° C. The mixture was stirred for 15 min, after which 4-(dimethylamino)cinnamaldehyde (0.2963 g, 1.69 mmol) was added. The reaction mixture was then stirred at room temperature for 24 h. The precipitated product was vacuum filtered and washed with small portions of water/ethanol to yield the desired extended chalcone as a red solid (0.4784 g, 92%). Melting point: 126.9-128.4° C. 1H NMR (400 MHz, CDCl3) δ 7.91 (d, J=7.8 Hz, 2H), 7.62 (dd, J=14.7, 10.9 Hz, 1H), 7.40 (d, J=8.4 Hz, 2H), 7.30 (d, J=7.9 Hz, 2H), 7.04-6.91 (m, 2H), 6.85 (dd, J=15.3, 10.8 Hz, 1H), 6.68 (d, J=8.4 Hz, 2H), 3.02 (s, 6H), 2.72 (q, J=7.6 Hz, 2H), 1.27 (t, J=7.6 Hz, 3H). 13 C NMR (101 MHz, CDCl3) δ 190.21, 151.18, 149.31, 146.05, 142.90, 136.48, 128.98, 128.64, 128.11, 124.41, 122.86, 122.68, 112.16, 40.33, 29.06, 15.38. Elemental anal.: calcd. C₂₁H₂₃NO: C, 82.58; H, 7.59; N, 4.59; O, 5.24; found C, 82.51; H, 7.71; N, 4.62.

### (2E,4E)-5-(4-(dimethylamino)phenyl)-1-(4-propylphenyl)penta-2,4-dien-1-one (Compound 5)

Red solid (0.3943 g, 83%). Melting point: 111.9-113.0° C. 1H NMR (400 MHz, CDCl3) δ 7.90 (d, J=7.1 Hz, 2H), 7.62 (dd, J=14.8, 10.8 Hz, 1H), 7.40 (d, J=8.3 Hz, 2H), 7.28 (s, 2H), 7.04-6.91 (m, 2H), 6.85 (dd, J=15.3, 10.9 Hz, 1H), 6.68 (d, J=8.3 Hz, 2H), 3.02 (d, J=1.5 Hz, 6H), 2.65 (t, J=7.7 Hz, 2H), 1.68 (h, J=7.4 Hz, 2H), 0.96 (t, J=7.3 Hz, 4H). 13C NMR (101 MHz, CDCl3) δ 190.20, 151.17, 147.79, 146.02, 142.89, 136.48, 130.61, 128.96, 128.70, 124.38, 123.90, 122.84, 112.14, 111.85, 40.30, 40.17, 38.15, 24.38, 13.91. Elemental anal.: calcd. C₂₂H₂₅NO: C, 82.72; H, 7.89; N, 4.38; O, 5.01; found C, 82.10; H, 7.82; N, 4.51.

### (2E,4E)-5-(4-(dimethylamino)phenyl)-1-(4-isopropylphenyl)penta-2,4-dien-1-one (Compound 6)

Orange solid (0.3968 g, 43%). Melting Point: 122.5-125.5° C. 1H NMR (400 MHz, CDCl3) δ 7.62 (dd, J=14.7, 10.8 Hz, 1H), 7.40 (d, J=8.8 Hz, 2H), 7.33 (d, J=8.2 Hz, 2H), 7.04-6.91 (m, 2H), 6.85 (dd, J=15.2, 10.9 Hz, 1H), 6.68 (d, J=8.8 Hz, 2H), 3.02 (s, 6H), 2.97 (q, J=6.9 Hz, 1H), 1.28 (d, J=6.9 Hz, 6H). 13C NMR (101 MHz, CDCl3) δ 190.12, 153.77, 145.91, 142.74, 136.52, 128.87, 128.56, 126.60, 122.75, 112.13, 40.29, 34.26, 23.76. Elemental anal.: calcd. C₂₂H₂₅NO: C, 82.72; H, 7.89; N, 4.38; O, 5.01; found C, 82.46; H, 7.94; N, 4.51.

### (2E,4E)-5-(4-(dimethylamino)phenyl)-1-(4-isobutylphenyl)penta-2,4-dien-1-one (Compound 7)

Orange solid (0.7069 g, 72%). Melting Point: 121.3-124.3° C. 1H NMR (400 MHz, CDCl3) δ 7.90 (d, J=8.3 Hz, 1H), 7.62 (dd, J=14.7, 10.9 Hz, 1H), 7.40 (d, J=8.9 Hz, 2H), 7.24 (d, J=8.2 Hz, 2H), 7.04-6.91 (m, 2H), 6.85 (dd, J=15.2, 10.9 Hz, 1H), 6.68 (d, J=8.9 Hz, 1H), 3.02 (s, 5H), 2.54 (d, J=7.2 Hz, 2H), 1.92 (dh, J=13.5, 6.8 Hz, 1H), 0.92 (d, J=6.6 Hz, 6H). 13 C NMR (101 MHz, CDCl3) δ 190.16, 151.06, 146.77, 145.95, 142.79, 136.43, 129.26, 128.89, 128.31, 122.82, 122.64, 112.13, 45.45, 40.27, 30.18, 22.40. Elemental anal.: calcd. C₂₂H₂₅NO: C, 82.84; H, 8.16; N, 4.20; O, 4.80; found C, 82.56; H, 8.10; N, 4.30.

### (2E,4E)-1-(4-cyclohexylphenyl)-5-(4-(dimethylamino)phenyl)penta-2,4-dien-1-one (Compound 8)

Orange solid (0.6055 g, 56%). Melting Point: 168.0-171.0° C. 1H NMR (400 MHz, CDCl3) δ 7.91 (d, J=8.4 Hz, 2H), 7.62 (dd, J=14.8, 10.8 Hz, 1H), 7.40 (d, J=8.8 Hz, 2H), 7.31 (d, J=8.3 Hz, 2H), 7.04-6.91 (m, 2H), 6.85 (dd, J=15.3, 10.8 Hz, 1H), 6.70 (d, J=8.4 Hz, 2H), 3.02 (s, 6H), 2.57 (td, J=8.5, 4.8 Hz, 1H), 1.95-1.82 (m, 4H), 1.81-1.73 (m, 1H), 1.53-1.37 (m, 4H), 1.38-1.21 (m, 1H). 13C NMR (101 MHz, CDCl3) δ 190.12, 152.95, 145.86, 142.70, 136.50, 128.86, 128.52, 126.99, 122.86, 122.67, 112.13, 77.24, 44.71, 40.29, 34.18, 26.79, 26.09. Elemental anal.: calcd. C₂₅H₂₉NO: C, 83.52; H, 8.13; N, 3.90; O, 4.45; found C, 83.51; H, 8.24; N, 3.99.

### (2E,4E)-1-(4-bromophenyl)-5-(4-(dimethylamino)phenyl)penta-2,4-dien-1-one (Compound 9)

Orange solid (0.7086 g, 70%). Melting Point: 175.3-178.3° C. 1H NMR (400 MHz, CDCl3) δ 7.83 (d, J=8.5 Hz, 2H), 7.68-7.57 (m, 3H), 7.40 (d, J=8.9 Hz, 2H), 6.95 (dd, J=20.9, 15.0 Hz, 2H), 6.84 (dd, J=15.3, 11.1 Hz, 1H), 6.68 (d, J=9.0 Hz, 1H), 3.03 (s, 6H). 13C NMR (101 MHz, CDCl3) δ 189.32, 151.17, 146.97, 143.69, 137.51, 131.76, 129.85, 129.05, 127.25, 122.31, 121.97, 112.09, 77.25, 40.26. Elemental anal.: calcd. C₁₉H₁₈BrNO: C, 64.06; H, 5.09; Br, 22.43; N, 3.93; O, 4.49; found C, 64.10; H, 5.13; N, 3.96.

### (2E,4E)-5-(4-(dimethylamino)phenyl)-1-(4-iodophenyl)penta-2,4-dien-1-one (Compound 10)

Orange solid (1.5643 g, 65%). Melting Point: 184.3-187.6° C. 1H NMR (400 MHz, CDCl3) δ 7.83 (d, J=8.5 Hz, 1H), 7.72-7.57 (m, 2H), 7.40 (d, J=8.9 Hz, 2H), 7.02-6.79 (m, 3H), 6.68 (d, J=8.9 Hz, 2H), 3.02 (s, 6H). 13C NMR (101 MHz, CDCl3) δ 189.60, 151.23, 147.00, 143.73, 138.07, 137.75, 129.78, 129.05, 124.09, 122.27, 121.90, 112.03, 99.88, 77.25, 40.21. Elemental anal.: calcd. C₁₉H₁₈INO: C, 56.59; H, 4.50; I, 31.47; N, 3.47; O, 3.97; found C, 53.57; H, 4.22; N, 3.11.

### (2E,4E)-5-(4-(dimethylamino)phenyl)-1-(4-(trifluoromethyl)phenyl)penta-2,4-dien-1-one (Compound 11)

Orange solid (0.7117 g, 72%). Melting point: 178.1-181.1° C. 1H NMR (400 MHz, CDCl3) δ 8.04 (d, J=8.1 Hz, 2H), 7.73 (d, J=8.1 Hz, 2H), 7.64 (dd, J=14.7, 11.1 Hz, 1H), 7.41 (d, J=8.9 Hz, 2H), 7.04-6.80 (m, 3H), 6.68 (d, J=8.9 Hz, 2H), 3.03 (s, 6H). 13C NMR (101 MHz, CDCl3) δ 189.54, 151.28, 147.69, 144.25, 141.71, 133.65, 129.16, 128.54, 125.53, 124.03, 122.45, 122.07, 112.06, 40.21. Elemental anal.: calcd. C₂₀H₁₈F₃NO: C, 69.56; H, 5.25; F, 16.50; N, 4.06; O, 4.63; found C, 69.47; H, 5.30; N, 4.06.

### (2E,4E)-5-(4-(dimethylamino)phenyl)-1-(4-nitrophenyl)penta-2,4-dien-1-one (Compound 12)

Brown solid (0.5024 g, 54%). Melting Point: 170.9-173.9° C. 1H NMR (400 MHz, CDCl3) δ 8.32 (d, J=8.8 Hz, 2H), 8.08 (d, J=8.8 Hz, 1H), 7.66 (dd, J=14.7, 11.2 Hz, 1H), 7.42 (d, J=8.9 Hz, 2H), 7.06-6.79 (m, 3H), 6.68 (d, J=8.9 Hz, 2H), 3.04 (s, 6H). 13C NMR (101 MHz, CDCl3) δ 188.83, 151.41, 149.75, 148.43, 144.99, 143.85, 130.52, 129.31, 129.16, 123.74, 121.95, 121.62, 112.03, 40.21. Elemental anal.: calcd. C₁₉H₁₈N₂O₃: C, 70.79; H, 5.63; N, 8.69; O, 14.89; found C, 70.79; H, 5.70; N, 8.67.

### 4-((2E,4E)-5-(4-(dimethylamino)phenyl)penta-2,4-dienoyl)benzonitrile (Compound 13)

Maroon solid (0.7700 g, 89%). Melting point: 165.1-168.2° C. 1H NMR (400 MHz, CDCl3) δ 8.02 (d, J=8.5 Hz, 1H), 7.77 (d, J=8.4 Hz, 1H), 7.65 (dd, J=14.7, 11.2 Hz, 1H), 7.41 (d, J=8.9 Hz, 2H), 7.01 (d, J=15.3 Hz, 1H), 6.92 (s, 1H), 6.91-6.80 (m, 2H), 6.68 (d, J=8.9 Hz, 2H), 3.03 (s, 6H). 13C NMR (101 MHz, CDCl3) δ 188.96, 151.40, 148.20, 144.80, 142.22, 132.36, 129.27, 128.65, 123.84, 121.95, 121.48, 118.26, 115.33, 111.99, 40.18. Elemental anal.: calcd. C₂₀H₁₈N₂O: C, 79.44; H, 6.00; N, 9.26; O, 5.29; found C, 77.70; H, 6.04; N, 9.00.

### (2E,4E)-1-(4-acetylphenyl)-5-(4-(dimethylamino)phenyl)penta-2,4-dien-1-one (Compound 14)

Orange solid (0.9028 g, 92%). Melting point: 134.2-137.2° C. 1H NMR (400 MHz, CDCl3) δ 8.08-7.98 (m, 4H), 7.64 (dd, J=14.7, 11.1 Hz, 1H), 7.41 (d, J=8.8 Hz, 2H), 7.03-6.81 (m, 3H), 6.68 (d, J=8.9 Hz, 2H), 3.03 (s, 6H), 2.66 (s, 3H). 13C NMR (101 MHz, CDCl3) δ 197.65, 189.89, 151.29, 147.46, 144.11, 142.34, 139.44, 129.14, 128.43, 124.00, 122.24, 122.19, 112.01, 40.19, 26.90. Elemental anal.: calcd. C₂₁H₂₁BrNO₂: C, 78.97; H, 6.63; N, 4.39; O, 10.02; found C, 78.29; H, 6.71; N, 4.45.

### (2E,4E)-5-(4-(dimethylamino)phenyl)-1-(4-methoxyphenyl)penta-2,4-dien-1-one (Compound 15)

Orange solid (0.8930 g, 73%). Melting Point: 137.0-140.0° C. 1H NMR (400 MHz, CDCl3) δ 7.99 (d, J=8.9 Hz, 2H), 7.62 (dd, J=14.7, 10.8 Hz, 1H), 7.40 (d, J=8.9 Hz, 2H), 7.05-6.91 (m, 3H), 6.91-6.79 (m, 1H), 6.68 (d, J=8.9 Hz, 2H), 3.88 (s, 3H), 3.02 (s, 6H). 13 C NMR (101 MHz, CDCl3) δ 188.82, 163.06, 151.03, 145.52, 142.54, 131.62, 130.54, 128.83, 124.38, 122.64, 122.53, 113.71, 112.09, 55.47, 40.26. Elemental anal.: calcd. C₂₀H₂₁NO₂: C, 78.15; H, 6.89; N, 4.56; O, 10.41; found C, 78.00; H, 7.00; N, 4.70.

### (2E,4E)-5-(4-(dimethylamino)phenyl)-1-(4-ethoxyphenyl)penta-2,4-dien-1-one (Compound 16)

Orange solid (0.7713, 83%). Melting Point: 145.8-148.8° C. 1H NMR (400 MHz, CDCl3) δ 7.98 (d, J=8.9 Hz, 1H), 7.62 (dd, J=14.7, 10.8 Hz, 1H), 7.40 (d, J=8.9 Hz, 2H), 7.05-6.90 (m, 3H), 6.85 (dd, J=15.3, 10.8 Hz, 1H), 6.68 (d, J=8.9 Hz, 2H), 4.11 (q, J=7.0 Hz, 2H), 3.02 (s, 5H), 1.45 (t, J=7.0 Hz, 3H). 13C NMR (101 MHz, CDCl3) δ 188.76, 162.48, 151.00, 145.40, 142.46, 131.39, 130.52, 128.79, 124.35, 122.62, 122.52, 114.13, 112.05, 63.69, 40.21, 14.71. Elemental anal.: calcd. C₂₁H₂₃NO₂: C, 78.47; H, 7.21; N, 4.36; O, 9.96; found C, 78.32; H, 7.13; N, 4.46.

### (2E,4E)-1-(benzo[d][1,3]dioxol-5-yl)-5-(4-(dimethylamino)phenyl)penta-2,4-dien-1-one (Compound 17)

Orange solid (0.7941, 80%). Melting point: 141.2-144.2° C. 1H NMR (400 MHz, CDCl3) δ 7.67-7.53 (m, 2H), 7.49 (d, J=1.7 Hz, 1H), 7.40 (d, J=8.9 Hz, 2H), 6.95 (d, J=14.3 Hz, 2H), 6.92-6.78 (m, 2H), 6.68 (d, J=8.9 Hz, 2H), 6.05 (s, 2H). 13C NMR (101 MHz, CDCl3) δ 188.33, 151.27, 148.15, 145.82, 142.77, 133.52, 130.33, 128.87, 124.24, 122.54, 122.31, 112.08, 108.37, 107.84, 101.75, 40.25. Elemental anal.: calcd. C₂₀H₁₉NO₃: C, 74.75; H, 5.96; N, 4.36; O, 14.93; found C, 74.75; H, 5.95; N, 4.40.

### (2E,4E)-5-(4-(dimethylamino)phenyl)-1-(4-(methylthio)phenyl)penta-2,4-dien-1-one (Compound 18)

Orange solid (0.7720 g, 81%). Melting point: 127.9-130.2° C. 1H NMR (400 MHz, CDCl3) δ 7.91 (d, J=8.5 Hz, 2H), 7.63 (dd, J=14.7, 10.9 Hz, 1H), 7.40 (d, J=8.9 Hz, 2H), 7.33-7.24 (m, 2H), 7.02-6.92 (m, 2H), 6.85 (dd, J=15.3, 10.9 Hz, 1H), 6.68 (d, J=8.9 Hz, 2H), 3.02 (s, 6H), 2.53 (s, 3H). 13C NMR (101 MHz, CDCl3) δ 189.23, 151.06, 146.07, 144.84, 142.97, 135.05, 128.91, 128.77, 125.11, 122.55, 122.35, 112.10, 40.27, 14.91. Elemental anal.: calcd. C₂₀H₂₁NOS: C, 74.27; H, 6.54; N, 4.33; O, 4.95; S, 9.91; found C, 74.20; H, 6.45; N, 4.44.

### (2E,4E)-1-(4-(1H-imidazol-1-yl)phenyl)-5-(4-(dimethylamino)phenyl)penta-2,4-dien-1-one (Compound 19)

Yellow solid (0.4769 g, 73%). Melting point: 200.7-203.7° C. 1H NMR (400 MHz, CDCl3) δ 8.11 (d, J=8.3 Hz, 2H), 7.97 (s, 1H), 7.68 (dd, J=14.7, 11.1 Hz, 1H), 7.51 (d, J=8.3 Hz, 2H), 7.42 (d, J=8.5 Hz, 2H), 7.37 (s, 1H), 7.00 (dd, J=15.0, 5.4 Hz, 2H), 6.88 (dd, J=15.2, 11.1 Hz, 1H), 6.70 (d, J=8.6 Hz, 2H), 3.04 (s, 6H). 13C NMR (101 MHz, CDCl3) δ 188.74, 151.26, 147.12, 143.91, 140.05, 137.54, 135.42, 130.96, 130.23, 129.09, 124.03, 122.20, 121.78, 120.74, 117.83, 112.02, 40.20. Elemental anal.: calcd. C₂₂H₂₁N₃O: C, 76.94; H, 6.16; N, 12.24; O, 4.66; found C, 76.79; H, 6.17; N, 12.25.

### (2E,4E)-5-(4-(dimethylamino)phenyl)-1-(3-(trifluoromethyl)phenyl)penta-2,4-dien-1-one (Compound 20)

Red solid (0.5782 g, 87%). Melting point: 112.3-115.3° C. 1H NMR (400 MHz, CDCl3) δ 8.24 (d, J=1.8 Hz, 1H), 8.17 (d, J=7.7 Hz, 1H), 7.85-7.78 (m, 1H), 7.75-7.59 (m, 2H), 7.44 (d, J=9.0 Hz, 2H), 7.08-6.84 (m, 3H), 6.71 (d, J=8.9 Hz, 2H), 3.05 (s, 6H). 13C NMR (101 MHz, CDCl3) δ 188.90, 151.29, 147.60, 144.24, 139.36, 131.41, 131.20, 130.87, 129.14, 128.63, 125.11, 124.01, 122.53, 122.14, 121.56, 112.03, 40.19. Elemental anal.: calcd. C₂₀H₁₈F₃NO: C, 69.56; H, 5.25; F, 16.50; N, 4.06; O, 4.63; found C, 69.27; H, 5.26; N, 4.08.

### (2E,4E)-1-(3-chlorophenyl)-5-(4-(dimethylamino)phenyl)penta-2,4-dien-1-one (Compound 21)

Red solid (0.5911 g, 65%). Melting point: 123.9-126.5° C. 1H NMR (400 MHz, CDCl3) δ 7.93 (t, J=1.9 Hz, 1H), 7.83 (d, J=7.8 Hz, 1H), 7.64 (dd, J=14.7, 11.1 Hz, 1H), 7.51 (ddd, J=7.9, 2.2, 1.1 Hz, 1H), 7.41 (dd, J=8.3, 5.6 Hz, 3H), 7.03-6.80 (m, 3H), 6.68 (d, J=8.9 Hz, 2H), 3.03 (s, 6H). 13C NMR (101 MHz, CDCl3) δ 189.17, 151.36, 147.37, 144.03, 140.53, 134.85, 132.25, 129.91, 129.20, 128.51, 126.44, 124.16, 122.32, 122.02, 112.13, 40.31. Elemental anal.: calcd. C₁₉HClNO: C, 73.19; H, 5.82; Cl, 11.37; N, 4.49; O, 5.13; found C, 73.06; H, 5.95; N, 4.54.

### (2E,4E)-5-(4-(dimethylamino)phenyl)-1-(2-(trifluoromethyl)phenyl)penta-2,4-dien-1-one (Compound 22)

Red solid (0.8021 g, 82%). Melting point: 113.9-116.9° C. 1H NMR (400 MHz, CDCl3) δ 7.74 (d, J=7.0 Hz, OH), 7.65-7.51 (m, 2H), 7.45-7.35 (m, 1H), 7.40-7.31 (m, 2H), 7.05 (ddd, J=15.3, 9.4, 1.0 Hz, 1H), 6.86-6.71 (m, 2H), 6.71-6.61 (m, 2H), 6.47 (d, J=15.3 Hz, 1H), 3.01 (s, 6H). 13C NMR (101 MHz, CDCl3) δ 195.19, 151.24, 149.51, 143.87, 139.47, 131.49, 129.40, 129.16, 128.15, 127.93, 127.61, 127.36, 126.58, 125.05, 121.86, 112.05, 40.22. Elemental anal.: calcd. calcd. C20H18F3NO: C, 69.56; H, 5.25; F, 16.50; N, 4.06; O, 4.63; found C, 69.84; H, 5.31; N, 4.15.

### (2E,4E)-5-(4-(dimethylamino)phenyl)-1-(o-tolyl)penta-2,4-dien-1-one (Compound 23)

Orange solid (0.3992 g, 60%). Melting Point: 76.7-78.6° C. 1H NMR (400 MHz, DMSO) δ 7.41 (td, J=8.7, 5.0 Hz, 4H), 7.30 (d, J=7.0 Hz, 2H), 7.15 (dd, J=15.1, 9.7 Hz, 1H), 7.05-6.89 (m, 2H), 6.70 (d, J=8.5 Hz, 2H), 6.61 (d, J=15.0 Hz, 1H), 2.96 (s, 6H), 2.31 (s, 3H). 13C NMR (101 MHz, CDCl3) δ 197.25, 151.23, 147.94, 143.08, 139.90, 136.54, 131.16, 130.62, 129.98, 129.07, 127.90, 127.71, 125.46, 122.34, 112.15, 40.33, 20.17. Elemental anal.: calcd. C₂₀H₂₁NO: C, 82.44; H, 7.26; N, 4.81; O, 5.49; found C, 82.32; H, 7.24; N, 4.97.

### (2E,4E)-1-(2-chlorophenyl)-5-(4-(dimethylamino)phenyl)penta-2,4-dien-1-one (Compound 24)

Orange solid (0.5252 g, 91%). Melting Point: 92.0-95.9° C. 1H NMR (400 MHz, CDCl3) δ 7.38 (tt, J=12.6, 7.9 Hz, 6H), 7.20 (dd, J=15.2, 10.3 Hz, 1H), 6.91-6.75 (m, 2H), 6.66 (d, J=8.5 Hz, 2H), 6.54 (d, J=15.2 Hz, 1H), 3.02 (s, 6H). 13C NMR (101 MHz, CDCl3) δ 194.10, 151.25, 148.48, 143.76, 139.68, 131.11, 130.84, 130.14, 129.14, 126.97, 126.69, 123.96, 122.09, 112.03, 40.22. Elemental anal.: calcd. C₁₉H₁₈ClNO: C, 73.19; H, 5.82; Cl, 11.37; N, 4.49; O, 5.13; found C, 72.92; H, 5.71; N, 4.47.

### (2E,4E)-1-(3,5-dichlorophenyl)-5-(4-(dimethylamino)phenyl)penta-2,4-dien-1-one (Compound 25)

Orange solid (0.4675 g, 82%). Melting Point: 152.5-155.5° C. 1H NMR (400 MHz, CDCl3) δ 7.81 (d, J=1.9 Hz, 2H), 7.65 (dd, J=14.7, 11.2 Hz, 1H), 7.52 (t, J=1.9 Hz, 1H), 7.41 (d, J=8.8 Hz, 2H), 7.01 (d, J=15.3 Hz, 1H), 6.91-6.79 (m, 2H), 6.69 (d, J=8.4 Hz, 2H), 3.03 (s, 6H). 13C NMR (101 MHz, CDCl3) δ 187.60, 151.42, 148.12, 144.76, 141.45, 135.48, 131.90, 129.33, 126.77, 124.03, 122.11, 121.21, 112.12, 40.29. Elemental anal.: calcd. C₁₉H₁₇Cl₂NO: C, 65.91; H, 4.95; Cl, 20.48; N, 4.05; O, 4.62; found C, 65.71; H, 5.02; N, 3.96.

### (2E,4E)-1-(2,4-dichlorophenyl)-5-(4-(dimethylamino)phenyl)penta-2,4-dien-1-one (Compound 26)

Orange solid (0.5911 g, 93%). Melting Point: 138.4-141.4° C. 1H NMR (400 MHz, CDCl3) δ 7.45 (d, J=1.9 Hz, 1H), 7.37 (dd, J=8.6, 2.4 Hz, 3H), 7.33 (d, J=2.0 Hz, 1H), 7.21 (dd, J=15.1, 10.7 Hz, 1H), 6.89 (d, J=15.3 Hz, 1H), 6.79 (dd, J=15.3, 10.8 Hz, 1H), 6.69-6.63 (m, 2H), 6.52 (d, J=15.1 Hz, 1H), 3.02 (s, 6H). 13C NMR (101 MHz, CDCl3) δ 192.76, 151.33, 148.72, 144.24, 138.08, 136.24, 132.18, 130.19, 130.03, 129.23, 127.11, 126.45, 123.87, 121.90, 112.01, 40.20. Elemental anal.: calcd. C₁₉H₁₇Cl₂NO: C, 65.91; H, 4.95; Cl, 20.48; N, 4.05; O, 4.62; found C, 66.01; H, 4.98; N, 3.91.

### (2E,4E)-1-(2-chloro-5-(trifluoromethyl)phenyl)-5-(4-(dimethylamino)phenyl)penta-2,4-dien-1-one (Compound 27)

Orange solid (0.5608 g, 80%). Melting Point: 138.4-140.7° C. 1H NMR (400 MHz, CDCl3) δ 7.70-7.60 (m, 2H), 7.56 (d, J=8.4 Hz, 1H), 7.38 (d, J=8.9 Hz, 2H), 7.22 (dd, J=15.1, 10.8 Hz, 1H), 6.91 (d, J=15.3 Hz, 1H), 6.81 (dd, J=15.3, 10.8 Hz, 1H), 6.66 (d, J=8.9 Hz, 2H), 6.53 (d, J=15.1 Hz, 1H), 3.03 (s, 6H). 13C NMR (101 MHz, CDCl3) δ 192.28, 151.44, 149.26, 144.77, 140.40, 134.98, 130.77, 129.57, 129.34, 129.23, 127.42, 127.38, 126.22, 125.96, 124.81, 123.72, 122.10, 121.73, 111.96, 40.16. Elemental anal.: calcd. C₂₀H₁₇ClF₃NO: C, 63.25; H, 4.51; Cl, 9.33; F, 15.01; N, 3.69; O, 4.21; found C, 63.15; H, 4.51; N, 3.56.

### (2E,4E)-5-(4-(dimethylamino)phenyl)-1-(2,5-dimethylphenyl)penta-2,4-dien-1-one (Compound 28)

Orange solid (0.4134 g, 59%). Melting point: 123.4-125.4° C. 1H NMR (400 MHz, CDCl3) δ 7.62-7.50 (m, 3H), 7.36 (d, J=8.3 Hz, 2H), 7.14 (s, 1H), 6.98-6.88 (m, 2H), 6.81 (dd, J=15.4, 10.7 Hz, 1H), 6.64 (d, J=8.3 Hz, 2H), 3.00-2.96 (m, 6H), 2.35 (s, 6H). 13C NMR (101 MHz, CDCl3) δ 197.33, 151.06, 147.59, 142.80, 139.74, 134.88, 133.19, 130.92, 130.61, 128.92, 128.28, 127.67, 122.35, 112.08, 40.25, 20.92, 19.58. Elemental anal.: calcd. C₂₁H₂₃NO: C, 82.58; H, 7.59; N, 4.59; O, 5.24; found C, 82.35; H, 7.77; N, 4.73.

### 4-(diethylamino)cinnamaldehyde (Compound 29)

Commercially available 4-(diethylamino)benzaldehyde (7.00 g, 40 mmol) was dis-solved in 30 mL of concentrated sulfuric acid under vigorous stirring. The solution was then cooled to 0° C. and acetaldehyde (5.2964 g, 120 mmol) was added dropwise within 3 h. After another hour, the reaction mixture was poured onto ice and neutralized with 20% NaOH. The precipitate was filtered, dried, and purified using flash column chromatography to yield the desired product as a dirty brown solid (1.3409 g, 16%). Melting Point: 71.5-74.5° C. 1H NMR (400 MHz, CDCl3) δ 9.58 (d, J=7.9 Hz, 1H), 7.47-7.39 (m, 2H), 7.36 (d, J=15.6 Hz, 1H), 6.70-6.61 (m, 2H), 6.52 (dd, J=15.6, 7.9 Hz, 1H), 3.42 (q, J=7.1 Hz, 4H), 1.20 (t, J=7.1 Hz, 6H). 13C NMR (101 MHz, CDCl3) δ 193.70, 153.98, 150.12, 130.85, 123.24, 120.99, 111.24, 44.55, 12.55. Elemental anal.: calcd. C₁₃H₁₇NO: C, 76.81; H, 8.43; N, 6.89; O, 7.87; found C, 77.32; H, 8.58; N, 6.56.

### 4-(piperidin-1-yl)cinnamaldehyde (Compound 30)

Commercially available 4-(piperidin-1-yl)benzaldehyde (3.0519 g, 16.12 mmol) was dissolved in 30 mL of concentrated sulfuric acid under vigorous stirring. The solution was then cooled to 0° C. and acetaldehyde (3.4131 g, 77.48 mmol) was added dropwise within 3 h. After another hour, the reaction mixture was poured onto ice and neutralized with 20% NaOH. The precipitate was filtered, dried, and purified using flash column chromatography to yield the desired product as a dirty brown solid (1.1506 g, 33%). Melting Point: 82.7-85.7° C. 1H NMR (400 MHz, CDCl3) δ 9.61 (d, J=7.8 Hz, 1H), 7.47-7.42 (m, 2H), 7.37 (d, J=15.7 Hz, 1H), 6.90-6.86 (m, 2H), 6.56 (dd, J=15.7, 7.9 Hz, 1H), 3.34 (t, J=5.2 Hz, 4H), 1.72-1.63 (m, 6H). 13C NMR (101 MHz, CDCl3) δ 193.75, 153.46, 130.40, 124.56, 123.27, 114.49, 48.81, 25.37, 24.32. Elemental anal.: calcd. C₁₄H₁₇NO: C, 78.10; H, 7.96; N, 6.51; O, 7.43; found C, 78.29; H, 8.07; N, 6.62.

### (2E,4E)-5-(4-(diethylamino)phenyl)-1-phenylpenta-2,4-dien-1-one (Compound 31)

Orange solid (0.8400 g, 49%). Melting Point: 103.9-106.9° C. 1H NMR (400 MHz, CDCl3) δ 7.94 (d, J=8.1 Hz, 2H), 7.66 (t, J=13.2 Hz, 1H), 7.44 (dt, J=25.2, 4.8 Hz, 4H), 7.04-6.91 (m, 2H), 6.85 (dd, J=15.0, 11.8 Hz, 1H), 6.67 (d, J=8.0 Hz, 2H), 3.43 (q, J=7.4 Hz, 4H), 1.22 (d, J=7.8 Hz, 6H). 13C NMR (101 MHz, CDCl3) δ 189.15, 148.78, 147.16, 143.85, 138.52, 137.18, 129.70, 129.37, 128.75, 123.23, 121.69, 121.59, 111.42, 44.49, 12.65. Elemental anal.: calcd. C₂₁H₂₂ClNO: C, 74.22; H, 6.52; Cl, 10.43; N, 4.12; O, 4.71; found C, 73.55; H, 6.67; N, 4.03.

### (2E,4E)-1-(4-chlorophenyl)-5-(4-(piperidin-1-yl)phenyl)penta-2,4-dien-1-one (Compound 33)

Orange solid (0.1883 g, 86%). Melting Point: 138.2-141.2° C. 1H NMR (400 MHz, CDCl3) δ 7.91 (d, J=8.2 Hz, 2H), 7.62 (dd, J=14.7, 10.9 Hz, 1H), 7.42 (dd, J=20.9, 8.2 Hz, 4H), 7.01-6.81 (m, 5H), 3.28 (t, J=5.4 Hz, 4H), 1.92-1.45 (m, 6H).

13C NMR (101 MHz, CDCl3) δ 189.15, 152.45, 146.63, 143.20, 138.68, 136.99, 129.73, 128.87, 128.80, 125.94, 123.13, 122.52, 115.18, 49.34, 25.50, 24.33. Elemental anal.: calcd. C₂₂H₂₂ClNO: C, 75.10; H, 6.30; Cl, 10.07; N, 3.98; O, 4.55; found C, 75.04; H, 6.37; N, 3.97.

### (2E,4E)-1-(4-chlorophenyl)-5-(4-(piperidin-1-yl)phenyl)penta-2,4-dien-1-one (Compound 34)

Orange solid (0.1318 g, 73%). Melting Point: 113.9-115.8° C. 1H NMR (400 MHz, CDCl3) δ 7.87-7.78 (m, 2H), 7.64 (dd, J=14.8, 11.0 Hz, 1H), 7.52 (s, 1H), 7.40 (d, J=8.5 Hz, 2H), 7.00 (d, J=15.3 Hz, 1H), 6.94-6.81 (m, 4H), 3.29 (t, J=5.3 Hz, 4H), 1.67 (dt, J=21.3, 5.2 Hz, 6H). 13C NMR (101 MHz, CDCl3) δ 187.73, 152.68, 147.90, 144.32, 141.39, 135.56, 132.02, 129.19, 126.82, 125.79, 122.95, 121.80, 115.18, 49.35, 25.61, 24.47. Elemental anal.: calcd. C₂₂H₂₁Cl₂NO: C, 68.4; H, 5.48; Cl, 18.35; N, 3.63; O, 4.14; found C, 66.94; H, 5.38; N, 3.35.

As used herein, “consisting essentially of” allows the inclusion of materials or steps that do not materially affect the basic and novel characteristics of the claim. Any recitation herein of the term “comprising”, such as in a claim or in a description of components of a composition or in a description of elements of a device, can be replaced as an alternative with “consisting essentially of” or “consisting of”.

While the present invention has been described in conjunction with certain preferred embodiments, one of ordinary skill, after reading the foregoing specification, will be able to effect various changes, substitutions of equivalents, and other alterations to the compositions and methods set forth herein. Any examples of descriptions of structures, components, materials, or method steps designed to perform a function or achieve a result are not intended to invoke “means plus function” or “step plus function” claim construction; the examples are merely provided to help illustrate the claimed subject matter using certain nonlimiting embodiments.

### REFERENCES

1. Scarpini, E.; Schelterns, P.; Feldman, H. Treatment of Alzheimer's disease; current status and new perspectives. Lancet Neurol. 2003, 2, 539-547.
2. Alzheimer's Association. Alzheimer's Facts and Figures Report; Alzheimer's Association: Chicago, IL, USA, 2022; www.alz.org/alzheimers-dementia/facts-figures.
3. Panza, F.; Seripa, D.; D'Onofrio, G.; Frisardi, V.; Solfrizzi, V.; Mecocci, P.; Pilotto, A. Neuropsychiatric Symptoms, Endophenotypes, and Syndromes in Late-Onset Alzheimer's Disease: Focus on APOE Gene. Int. J. Alzheimer's Dis. 2011, 2011, 721457.
4. Murphy, M. P.; LeVine, H., 3rd. Alzheimer's Disease and the Amyloid-13 Peptide. J. Alzheimer's Dis. 2010, 19, 311-323.
5. Quiroz, J. P.; Zeng, A.; Young, M.; Gordon, P.; Jaipuria, A.; Reed, K. J.; Landry, G. M.; Yang, S.; Asher, S.; Zhang, S. R. C.; et al. Homotaurine and Curcumin Analogues as Potential Anti-Amyloidogenic Agents. Chemistry 2023, 5, 223-241.
6. Müller, U. C.; Zheng, H. Physiological Functions of APP Family Proteins. Cold Spring Harb. Perspect. Med. 2012, 2, a006288.
7. Chen, G. F.; Xu, T. H.; Yan, Y.; Zhou, Y. R.; Jiang, Y.; Melcher, K.; Xu, H. E. Amyloid beta: Structure, biology and structure-based therapeutic development. Acta Pharmacol. Sin. 2017, 38, 1205-1235.
8. Tiwari, S.; Atluri, V.; Kaushik, A.; Yndart, A.; Nair, M. Alzheimer's disease: Pathogenesis, diagnostics, and therapeutics. Int. J. Nanomed. 2019, 14, 5541-5554.
9. Olsson, F.; Schmidt, S.; Althoff, V.; Munter, L. M.; Jin, S.; Rosqvist, S.; Lendahl, U.; Multhaup, G.; Lundkvist, J. Characterization of Intermediate Steps in Amyloid Beta (A13) Production under Near-native Conditions. J. Biol. Chem. 2014, 289, 1540-1550.
10. Madav, Y.; Wairkar, S.; Prabhakar, B. Recent therapeutic strategies targeting beta amyloid and tauopathies in Alzheimer's disease. Brain Res. Bull. 2019, 146, 171-184.
11. Takami, M.; Nagashima, Y.; Sano, Y.; Ishihara, S.; Morishima-Kawashima, M.; Funamoto, S.; Ihara, Y. γ-Secretase: Successive Tripeptide and Tetrapeptide Release from the Transmembrane Domain of 13-Carboxyl Terminal Fragment. J. Neurosci. 2009, 29, 13042-13052.
12. Roeters, S. J.; Iyer, A.; Pletikapic', G.; Kogan, V.; Subramaniam, V.; Woutersen, S. Evidence for Intramolecular Antiparallel Beta-Sheet Structure in Alpha-Synuclein Fibrils from a Combination of Two-Dimensional Infrared Spectroscopy and Atomic Force Microscopy. Sci. Rep. 2017, 7, 41051.
13. Nussbaum, J. M.; Schilling, S.; Cynis, H.; Silva, A.; Swanson, E.; Wangsanut, T.; Tayler, K.; Wiltgen, B.; Hatami, A.; Rönicke, R.; et al. Prion-like behaviour and tau-dependent cytotoxicity of pyroglutamylated amyloid-β. Nature 2012, 485, 651-655.
14. Shipton, O. A.; Leitz, J. R.; Dworzak, J.; Acton, C. E. J.; Tunbridge, E. M.; Denk, F.; Dawson, H. N.; Vitek, M. P.; Wade-Martins, R. W.; Paulsen, O.; et al. Tau Protein Is Required for Amyloid-Induced Impairment of Hippocampal Long-Term Potentiation. J. Neurosci. 2011, 31, 1688-1692.
15. Chen, L.-L.; Fan, Y.-G.; Zhao, L.-X.; Zhang, Q.; Wang, Z.-Y. The metal ion hypothesis of Alzheimer's disease and the anti-neuroinflammatory effect of metal chelators. Bioorg. Chem. 2022, 131, 106301.
16. Kumar, D.; Ganeshpurkar, A.; Kumar, D.; Modi, G.; Gupta, S. K.; Singh, S. K. Secretase inhibitors for the treatment of Alzheimer's disease: Long road ahead. Eur. J. Med. Chem. 2018, 148, 436-452.
17. de la Torre, J. C.; Gonzalez-Lima, F. The FDA Approves Aducanumab for Alzheimer's Disease, Raising Important Scientific Questions 1. J. Alzheimer's Dis. 2021, 82, 881-882.
18. Verger, A.; Yakushev, I.; Albert, N. L.; van Berckel, B.; Brendel, M.; Cecchin, D.; Fernandez, P. A.; Fraioli, F.; Guedj, E.; Morbelli, S.; et al. FDA approval of lecanemab: The real start of widespread amyloid PET use?—The EANM Neuroimaging Committee perspective. Eur. J. Nucl. Med. Mol. Imaging 2023, 50, 1553-1555.
19. SwissADME. www.swissadme.ch/index.php (accessed on 15 Mar. 2024).
20. Alvarez, J.; Alvarez-Illera, P.; Santo-Domingo, J.; Fonteriz, R. I.; Montero, M. Modeling Alzheimer's Disease in Caenorhabditis elegans. Biomedicines 2022, 10, 288.
21. Miller, D. E.; Cook, K. R.; Arvanitakis, A. V.; Hawley, R. S. Third Chromosome Balancer Inversions Disrupt Protein-Coding Genes and Influence Distal Recombination Events in Drosophila melanogaster. G3 2017, 6, 1959-1967, Correction in G3 2017, 7, 3837.
22. Sekiya, M.; Iijima, K. M. Phenotypic analysis of a transgenic Drosophila model of Alzheimer's amyloid-3 toxicity. STAR Protoc. 2022, 2, 100501.
23. Liu, C.; Zhou, H.; Sheng, X. B.; Liu, X. H.; Chen, F. H. Design, synthesis and SARs of novel telomerase inhibitors based on BIBRA1532. Bioorg Chem. 2020, 102, 104007.
24. Ono, M.; Doi, Y.; Watanabe, H.; Ihara, M.; Ozaki, A.; Saji, H. Structure-activity relationships of radioiodinated diphenyl derivatives with different con-jugated double bonds as ligands for α-synuclein aggregates. RSC Adv. 2016, 6, 44305-44312.
25. Niu, C.; Tuerxuntayi, A.; Li, G.; Kabas, M.; Dong, C.-Z.; Aisa, H. A. Design, synthesis and bioactivity of chalcones and its analogues. Chin. Chem. Lett. 2017, 28, 1533-1538.
26. Le, V.-H.; Lien, V. T. K.; Pham, V. T.; Tran, Q. T.; Thuy, P. T.; Ha, C. V.; Doanh, V. V.; Ha, L. T.; Hanh, C. H.; Thao, P. N.; et al. Effect of extended rr-conjugation on photophysical characteristics of chalcone and cinnamyli-deneacetophenone. Mater. Sci. Semicond. Process. 2023, 162, 107507.
27. Alphonsa, A. T. (E)-1-(benzo [d][1,3]dioxol-5-yl)-3-phenyl prop 2-en-1-ones: Design, synthesis, anti-cancer activity and molecular docking studies. Chem. Data Coll. 2020, 25, 100310.
28. Zhou, B.; Jiang, P.; Lu, J.; Xing, C. Characterization of the Fluorescence Properties of 4-Dialkyaminochalcones and Investigation of the Cytotoxic Mechanism of Chalcones. Arch. Pharm. 2016, 349, 539-552.

1. A compound of any of Formulas I-V below: 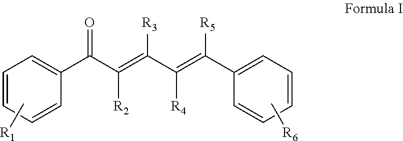
wherein
R1 is one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof;
R2, R3, R4, and R5 are independently hydrogen or a substituent; and
R6 is dialkylamino with each alkyl portion having C1-C5, or the alkyl portions fused to form a 5- or 6-membered saturated ring, or hydrogen; 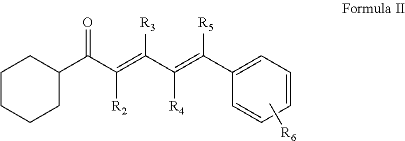
wherein
R2, R3, R4, and R5 are independently hydrogen or a substituent; and
R6 is dialkylamino with each alkyl portion having C1-C5, or the alkyl portions fused to form a 5- or 6-membered saturated ring, or hydrogen; 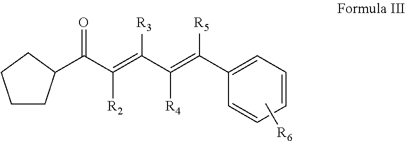
wherein
R2, R3, R4, and R5 are independently hydrogen or a substituent; and
R6 is dialkylamino with each alkyl portion having C1-C5, or the alkyl portions fused to form a 5- or 6-membered saturated ring, or hydrogen; 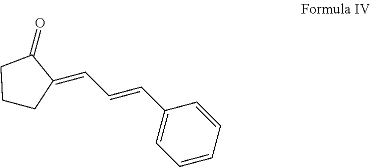 [SMILES: O=C1CCC/C1=C\C=C\c1ccccc1]
wherein any position not indicated with H can be independently any substituent; 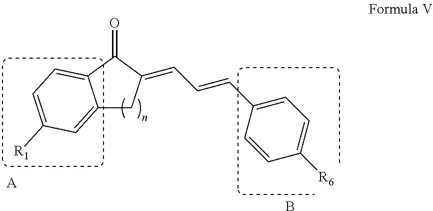
wherein
R1 is one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof;
R6 is dialkylamino with each alkyl portion having C1-C5, or the alkyl portions fused to form a 5- or 6-membered saturated ring, or hydrogen; and
n=0, 1, 2, or 3.

2. The compound of claim 1, wherein the compound is a compound of Formula VI below: 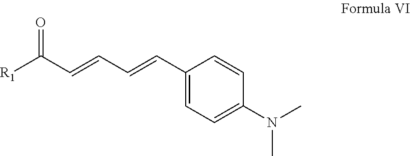
wherein R1 is an aromatic ring with one or two halogens at the para, meta, or di-meta position, one or two methyl groups at the para, meta, or di-meta position, one or two halomethyl, dihalomethyl, or trihalomethyl groups at the para, meta, or di-meta position; cyano, nitro, C1-C5 alkyloxy, C1-C5 keto, C1-C5 thioether, C1-C6 alkyl or cycloalkyl, hydrogen, another substituent, or any combination thereof.

3. The compound of claim 2, wherein the compound is selected from the group consisting of Compounds 4-7, 9-10, and 13-28.

4. The compound of claim 3, wherein the compound is selected from the group consisting of Compounds 4-5, 7, 13-14, 18-22, and 25-27.

5. The compound of claim 1, wherein the compound is not any of Compounds 6, 9, 10, 15, 16, 17, 23, 24, or 28.

6. The compound of claim 1, wherein the compound is selected from the group consisting of Compounds 32-34.

7. The compound of claim 1, wherein the compound inhibits aggregation of an amyloid precursor, such as Aβ 1-42.

8. The compound of claim 1, wherein the compound inhibits amyloid plaque formation in a mammalian brain, such as in a human brain.

9. The compound of claim 1 which can cross a blood-brain barrier of a living mammal.

10. A pharmaceutical composition comprising a compound of claim 1 and one or more excipients.

11. A method to aid in preventing formation or re-formation of amyloid plaque in a mammalian subject's brain, the method comprising administering an effective amount of a compound of claim 1 to the mammalian subject.

12. The method of claim 11, wherein the subject was previously treated to remove amyloid plaques from its brain.

13. The method of claim 12, wherein the previous treatment comprises administration of an antibody having a binding specificity for an epitope found in amyloid plaque.

14. The method of claim 13, wherein the antibody is selected from the group consisting of aducanumab, lecanemab, and donancinab.

15. The method of claim 11, wherein amyloid plaque formation in the subject's brain is inhibited to any extent.

16. The method of claim 11, wherein the subject is a human patient who has Alzheimer's Disease.

17. A method to aid in treating Alzheimer's Disease, the method comprising:
(a) administering to a human patient who has Alzheimer's Disease an antibody having a binding specificity for an epitope found in amyloid plaque; and
(b) administering an effective amount of a compound of claim 1 to said patient.

18. The method of claim 17, wherein steps (a) and (b) are performed sequentially or concurrently.

19. The method of claim 17, whereby reoccurrence of amyloid plaque formation in the patient is inhibited compared to a method comprising only step (a).

20. The method of claim 17, wherein the antibody administered in step (a) is selected from the group consisting of aducanumab, lecanemab, and donancinab.

21. A kit comprising:
(i) a compound of claim 1; and
(ii) an antibody having a binding specificity for an epitope found in amyloid plaque.

22. The kit of claim 21, where the antibody is selected from the group consisting of aducanumab, lecanemab, and donancinab.
